(19) United States
(12) Patent Application Publication
(10) Pub. No.: US 2005/0191628 A1
(43) Pub. Date: Sep. 1, 2005

(54) Antibiotics based upon bacteriophage lysis proteins

(76) Inventors: Ryland F. Young, College Station, TX (US); William D. Roof, College Station, TX (US); Douglas K. Struck, College Station, TX (US); Thomas G. Bernhardt, College Station, TX (US); Ing-Nang Wang, College Station, TX (US)

Correspondence Address: FULBRIGHT & JAWORSKI, LLP, 1301 MCKINNEY, SUITE 5100, HOUSTON, TX, 77010-3095, US

(21) Appl. No.: 10/625,357

(22) Filed: Jul. 23, 2003

Related U.S. Application Data
(62) Division of application No. 09/626,036, filed on Jul. 27, 2000, now abandoned.
(60) Provisional application No. 60/146,455, filed on Jul. 30, 1999.

(51) Int. Cl. C12Q 1/68; C12N 15/74; C12N 1/21

(52) U.S. Cl. 435/6; 435/471; 435/252.3

(57) ABSTRACT

This invention relates to polypeptide antibiotics, including materials and methods related thereto, based upon the observation that bacteriophage elaborate proteins that cause host cell lysis by interfering with specific steps in cell wall biosynthesis. Examples of antibiotics based upon this invention include the bacteriophage φX174 gene E product and structurally and/or functionally related polypeptide and small protein antibiotics that interact with MraY, and the bacteriophage Qβ gene A₂ product and structurally and/or functionally related polypeptide and small protein antibiotics that interact with MurA. This leads to the general model for obtaining new polypeptide antibiotics by using genetic approaches based on these findings to find polypeptide sequences which cause bacterial cell lysis.

This application claims priority to the U.S. Provisional Application No. 60/146,455 filed Jul. 30, 1999.

This work herein was supported by grants from the United States Government. The Untied States Government may have certain rights in the invention.

### FIELD OF THE INVENTION

This invention relates to polypeptide antibiotics, including materials and methods related thereto, or to synthetic antibiotic compounds modeled to function like polypeptide antibiotics.

### BACKGROUND OF THE INVENTION

I. Bacteriophage Lysis

At the end of the infective cycle, most bacterial viruses, or phages, destroy the host cell to achieve dispersal of the progeny virions. This process is called lysis. The lysis of a bacterial cell requires destroying or otherwise compromising the cell wall, or peptidoglycan, a polymer of amino-sugars crosslinked with oligopeptides which surrounds the cell outside the cytoplasmic membrane. Complex phages with double-stranded DNA genomes use a multigene system for achieving host lysis. These systems feature a muralytic enzyme that degrades the cell wall and other proteins involved in the export of the enzyme across the membrane, its regulation and activation and degradation events ancillary to peptidoglycan degradation. In contrast, small lytic phages, with constrained genome sizes, use a single-gene lysis strategy. Remarkably, in these phages, the single lysis gene does not encode a muralytic enzyme, raising the issue of how lysis is achieved if no enzyme is directed against the host cell wall. Two examples of small phages with single-gene lysis systems are: bacteriophage φX174, the prototype for the single-stranded circular DNA phage class Microviridae; and bacteriophage Qβ, a representative of the group III single-stranded RNA phage (FIG. 1). φX174 has 10 genes in its 5.7 kb genome; the single lysis gene is E, a 91 codon cistron embedded in the +1 reading frame within another essential and larger phage gene, D. Qβ has only 3 genes in its 4.1 kb ssRNA genome: A₂, coat and replicase. Replicase is involved in replication of the RNA genome, and Coat is the major constituent of the virus particle. One copy of the A₂ protein, also called the maturation protein, is present in the virus particle and is required for the ability of the particle to adsorb to its biological target, the F pilus of male bacteria. A second function of the A₂ protein is host lysis. In both single-gene systems, cloning experiments have shown that expression of the lysis gene, E or A₂, is necessary and sufficient for host cell lysis, irrespective of the other virus genes. Although many models have been proposed, the basic mechanism by which these single-gene lysis systems elicit host lysis is unknown. Single-gene lysis has the same cell growth requirements as lysis mediated by cell wall synthesis inhibitors, like penicillin. Moreover, the lytic lesions resulting from E expression localize to the growing cell septum, a site of concentrated cell wall synthesis.

II. Peptidoglycan Biosynthesis in Bacteria

Most eubacteria have a cell wall based on a conserved peptidoglycan structure in which glycan strands made up of alternating N-acetylglucosamine-N-acetylmuramic acid (NAG-NAM) diaminosaccharide polymers linked with glycosidic bonds and cross-linked with an pentapeptide, or related oligopeptide, of conserved sequence. The biosynthetic pathway consists of a number of cytoplasmic steps (FIG. 2). The first committed step in peptidoglycan synthesis is the conversion of UDP-NAG to UDP-NAG-enolpyruvate, catalyzed by UDP-NAG carboxyvinyltransferase, the product of the murA gene in E. coli. Further steps are catalyzed by cytoplasmic enzymes, resulting in UDP-NAM-pentapeptide, the final cytoplasmic precursor. This precursor and the lipid undecaprenolphosphate are the substrates of MraY, a membrane-embedded enzyme which catalyzes the formation of the first lipid-linked precursor, undecaprenol-NAM-pentapeptide. Another enzymatic step results in the donation of NAG from UDP-NAG, resulting in the formation of the last intracellular precursor, undecaprenol-NAM-pentapeptide-NAG. This precursor is exported to the outer surface of the membrane where undecaprenol-linked higher oligomers are formed and then incorporated into the polymeric peptidoglycan by a multi-enzyme complex including a number of penicillin-binding proteins (PBPs) located in the periplasm of E. coli.

Until the present invention, the target proteins of the single-gene lysis proteins were unknown. For both E and A₂, a combination of genetic and biochemical approaches were used to ascertain the target of the lysis proteins. The combination of genetic and biochemical analysis has demonstrated unequivocally that the steps catalyzed by the conserved enzymes MraY and MurA of the peptidoglycan biosynthesis pathway are the targets of the lysis proteins E and A₂, respectively.

Thus, this invention utilizes polypeptides to cause bacterial lysis by inhibiting cell wall synthesis, in a manner similar to fungal anti-cell wall antibiotics like penicillin. However, unlike fungal antibiotics, proteins can be easily engineered by recombinant DNA technology and are also subjects for modern molecular genetic analysis. Thus, this invention is indeed novel in that it is the first time that peptide antibiotics are designed based on the fact that the lysis polypeptides inhibit enzymes involved in bacterial cell wall synthesis.

### SUMMARY OF THE INVENTION

This invention relates to polypeptide antibiotics, including materials and methods related thereto, wherein the polypeptide antibiotics inhibit steps in cell wall synthesis or synthesis of other envelope components essential for the integrity of the cell, thereby causing lysis upon cell division or continued growth in the absence of cell wall synthesis. More particularly, the present invention relates to antibiotics, and methods related thereto, based upon the novel observation that genes E of phage φX174 and A₂ of phage Qβ encode polypeptide products which inhibit bacterial cell wall biosynthesis at distinct enzymatic steps, those encoded by MraY and MurA, respectively. Such antibiotics can include, but are not limited to, protein and/or polypeptide antibiotics related to the products of gene E and A₂. The finding that two different lytic bacteriophages target different steps in cell wall biosynthesis leads to the concept that other bacteriophages using a single-gene to accomplish host cell lysis will encode different proteins to attack other steps of cell wall biosynthesis. This leads to a general method for finding new phage-encoded polypeptides specific for these or other steps in cell wall biosynthesis or synthesis of other envelope components essential for the integrity of the cell.

A specific embodiment of the present invention is a method of screening for a candidate bacterial nucleic acid sequence that encodes a target polypeptide for a single-gene lysis polypeptide comprising: contacting bacteria with the lysis polypeptide; selecting for bacterial survivors of cell lysis caused by the lysis polypeptide that survive lysis by having a candidate bacterial nucleic acid sequence that encodes a target polypeptide making cells resistant to lysis by the lysis polypeptide; and mapping the candidate bacterial nucleic acid sequence, wherein the mapped sequence corresponds to the nucleic acid sequence which encodes the target polypeptide. A skilled artisan will realize that any bacterial protein that is involved in cell wall biosynthesis or synthesis of other envelope components essential for the integrity of the cell may be a target polypeptide encoded by a candidate bacterial nucleic acid sequence.

In yet another specific embodiment, contacting the bacteria with the lysis polypeptide comprises transforming bacteria with a vector comprising a nucleic acid sequence that encodes a single-gene lysis polypeptide. Further, the lysis polypeptide may be contacted with the bacteria by inducing the expression of the lysis polypeptide.

In another specific embodiment, the vector comprises a mutated lysis polypeptide. A skilled artisan will recognize that mutation of the lysis polypeptide may comprise modifying the amino acid sequence of the polypeptide or the nucleic acid sequence encoding the polypeptide. Mutagenesis may be performed using standard techniques well known in the art, including, but not limited to, chemical mutagenesis, radiation mutagenesis, truncation of amino acids, site-directed mutagenesis, transposon mutagenesis or spontaneous mutagenesis.

In a further embodiment, the mapped bacterial nucleic acid sequence may be isolated. Further, the characteristics of the isolated bacterial nucleic acid sequence may be determined. Determining the characteristics of the nucleic acid sequence may comprise gel electrophoresis or nucleic acid sequence analysis.

In yet another specific embodiment, the mapped bacterial nucleic acid sequence may be inserted into an expression vector to produce a polypeptide. One skilled in the art will recognize that this is a standard technique to utilize bacteria to produce large quantities of a protein for isolation and purification. Thus, the polypeptide may be isolated from the expression vector to determine the characteristics associated with the polypeptide. The characteristics may be determined using standard methods that include, but are not limited to, electrophoresis, spectrophotometric analysis, amino acid analysis, structural analysis or analysis of biochemical functions.

In another specific embodiment, the bacteria may comprise a vector comprising a nucleic acid sequence encoding a polypeptide involved in cell wall synthesis or synthesis of other envelope components essential for the integrity of the cell.

A specific embodiment of the present invention is a method of screening for a bacteriophage lysis polypeptide that targets bacterial cell wall synthesis or synthesis of other envelope components essential for the integrity of the cell comprising: obtaining a panel of recombinant bacterial strains, each overexpressing at least one recombinant nucleic acid sequence encoding a target polypeptide involved in cell wall synthesis or synthesis of other envelope components essential for the integrity of the cell, or a non-target polypeptide as a control; obtaining a candidate bacteriophage; contacting the panel of recombinant bacterial strains with the candidate bacteriophage; selecting for bacteriophage that is lysis-defective on at least one recombinant bacterial strain, wherein said bacteriophage expresses a single-gene lysis polypeptide that interacts with a target polypeptide involved in cell wall synthesis or synthesis of other envelope components essential for the integrity of the cell; and mapping a nucleic acid sequence in the bacteriophage, wherein the nucleic acid sequence encodes a single-gene lysis polypeptide. Exemplary sources of a candidate bacteriophage include, but may not be limited to, animal digestive tracts, fecal matter, sewage, waste water, natural salt water, fresh water and soil. Further the panel of bacteria strains may comprise Gram-negative bacteria, Gram-positive bacteria or a combination of Gram-negative and Gram-positive bacteria. In yet another aspect of the invention, the bacteriophage nucleic acid sequence may be isolated and characterized. The sequence may be characterized using techniques that are known and well used in the art including, but not limited to, gel electrophoresis or nucleic acid sequence analysis.

In yet a further embodiment, the panel of recombinant bacterial strains further comprises at least one mutated target polypeptide. The mutated target polypeptide may comprise modification of the amino acid sequence of the polypeptide or the nucleic acid sequence encoding the polypeptide. Modification can utilize standard mutagenesis techniques including, but not limited to, chemical mutagenesis, radiation mutagenesis, truncation of amino acids, spontaneous mutagenesis, transposon mutagenesis or site-directed mutagenesis.

Another specific embodiment of the present invention is a method of screening for nucleic acid sequences which encode a single-gene lysis polypeptide comprising: obtaining a library of DNA sequences cloned into an inducible plasmid expression vector; transforming the library into a bacterial strain; contacting the bacterial strain with polypeptides produced from the library after induction; selecting for vector plasmids that produce lysis polypeptides, wherein the vector plasmids are released into the medium after cell lysis; and determining the nucleic acid sequence encoding the lysis polypeptide from the plasmid DNA isolated from the lysed cells.

In specific embodiments, the library of DNA sequences comprises libraries constructed from bacterial chromosomal DNA, plasmid DNA from Gram positive bacteria, plasmid DNA from Gram negative bacteria or DNA pooled from uncharacterized bacteriophages. A skilled artisan will recognize that the DNA libraries may be constructed from genomic DNA or cDNA. Specifically, the cDNA library may be constructed from RNA bacteriophages. The uncharacterized bacteriophages may be isolated from the sources selected from the group consisting of animal digestive tracts, fecal matter, sewage, waste water, natural salt water, fresh water and soil.

A specific embodiment of the present invention is a method of screening for a bacteriophage, wherein the bacteriophage has enhanced lytic activity comprising: obtaining a recombinant bacterial strain, wherein the bacterial strain is transformed with a vector comprising a nucleic acid sequence encoding a recombinant target polypeptide involved in cell wall synthesis or synthesis of other envelope components essential for the integrity of the cell; obtaining a candidate bacteriophage; contacting the recombinant bacterial strain with the candidate bacteriophage; selecting for survivor bacteriophages; and mapping the bacteriophage nucleic acid sequence which encodes the single-gene lysis polypeptide. Specific examples of the target polypeptide include, but are not limited to, MurA or MraY.

In a further specific embodiment, the recombinant bacterial strain comprises a mutated target polypeptide. Particularly, the mutated target polypeptide comprises modifying the amino acid sequence of the polypeptide or the nucleic acid sequence encoding the polypeptide. One skilled in the art will recognize that the target polypeptide may be mutated using any of the various mutagenesis techniques that are well known in the art.

Specific embodiments of the present invention include a polypeptide antibiotic comprising at least an amino acid sequence or derivative thereof that interacts with a protein involved in cell wall synthesis or synthesis of other envelope components essential for the integrity of the cell. More particularly, interaction with the protein inhibits cell wall synthesis or synthesis of other envelope components essential for the integrity of the cell. One skilled in the art will recognize that the polypeptide antibiotic may be produced from a biological or a synthetic source. Further, the polypeptide antibiotic includes, but is not limited to, peptide fragments or derivatives (e.g., mutations) thereof which interact with a protein involved in cell wall synthesis or synthesis of other envelope components essential for the integrity of the cell.

Another specific embodiment of the present invention is a method of polypeptide antibiotic killing comprising: contacting a bacterium with a single-gene lysis polypeptide antibiotic, wherein the antibiotic inhibits a target protein involved in cell wall synthesis or synthesis of other envelope components essential for the integrity of the cell, leading to cell lysis upon cell division or continued cell growth. Exemplary target proteins include, but are not limited to, MurA or MraY. Further, specific polypeptide antibiotics may be a bacteriophage φX174 E gene product or a bacteriophage Qβ A₂ gene product. More particularly, the antibiotic may be selected from the group consisting of the bacteriophage φX174 E gene product, a fragment of the E gene product, a derivative of the E gene product, or a protein that is homologous or analogous to the E gene product. Yet further, the antibiotic may be selected from the group consisting of the bacteriophage Qβ A₂ gene product, a fragment of the A₂ gene product, a derivative of the A₂ gene product, or a protein that is homologous or analogous to the A₂ gene product.

In yet further specific embodiments, a polypeptide antibiotic comprises at least a portion of the E gene product which portion interacts with bacterial MraY. Specifically, the antibiotic may be the E gene product. Yet further, the polypeptide antibiotic may comprise the portion of the E gene product which interacts with bacterial MraY and may be selected from the group consisting of: at least a portion of the bacteriophage φX174 E gene product, at least a portion of a fragment of the E gene product, at least a portion of a derivative of the E gene product, or at least a portion of a polypeptide that is homologous or analogous to a portion of the E gene product that interacts with bacterial MraY.

In another embodiment, a polypeptide antibiotic comprises at least a portion of the A₂ gene product which portion interacts with bacterial MurA. Specifically, the antibiotic may be the gene A₂ gene product. Yet further, the polypeptide antibiotic may comprise the portion of the A₂ gene product which interacts with bacterial MurA and may be selected from the group consisting of: at least a portion of the bacteriophage Qβ A₂ gene product, at least a portion of a fragment of the A₂ gene product, at least a portion of a derivative of the A₂ gene product, or at least a portion of a polypeptide that is homologous or analogous to a portion of the A₂ gene product that interacts with bacterial MurA.

A specific embodiment is a polypeptide antibiotic comprising at least a sequence that interacts with MraY. Specifically, the polypeptide antibiotic interacts with MraY to inhibit the MraY activity. Further, the sequence that interacts with MraY may be selected from the group consisting of the bacteriophage φX174 E gene product, a fragment of the E gene product, a derivative of the E gene product, or a protein that is homologous or analogous to the E gene product.

Another specific embodiment is a polypeptide antibiotic comprising at least a sequence that interacts with MurA. More particularly, the polypeptide antibiotic interacts with MurA to inhibit the MurA activity. Yet further, the sequence that interacts with MurA may be selected from the group consisting of the bacteriophage Qβ A₂ gene product, a fragment of the A₂ gene product, a derivative of the A₂ gene product, or a protein that is homologous or analogous to the A₂ gene product.

As used herein the specification, “a” or “an” may mean one ore more. As used herein the claim(s), when used in conjunction with the word “comprising”, the words “a” or “an” may mean one or more than one. As used herein “another” may mean at least a second or more.

Other and further objects, features and advantages would be apparent and eventually more readily understood by reading the following specification and by reference to the accompanying drawings forming a part thereof, or any examples of the presently preferred embodiments of the invention are given for the purpose of the disclosure.

### BRIEF DESCRIPTION OF THE DRAWINGS

FIG. 1 shows the two examples of small phages bacteriophages with single-gene lysis systems.

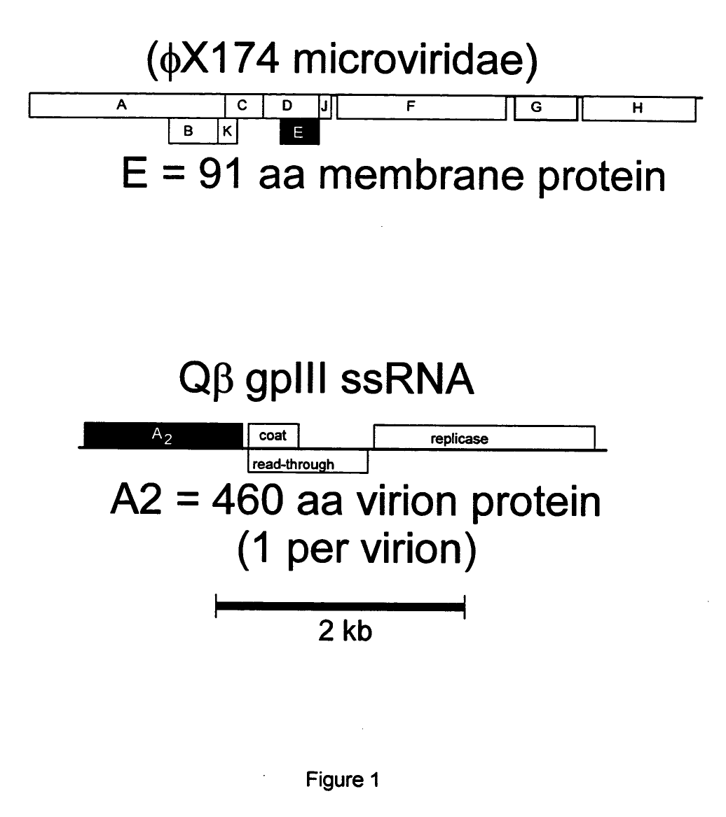

FIG. 2 shows the peptidoglycan biosynthesis pathway.

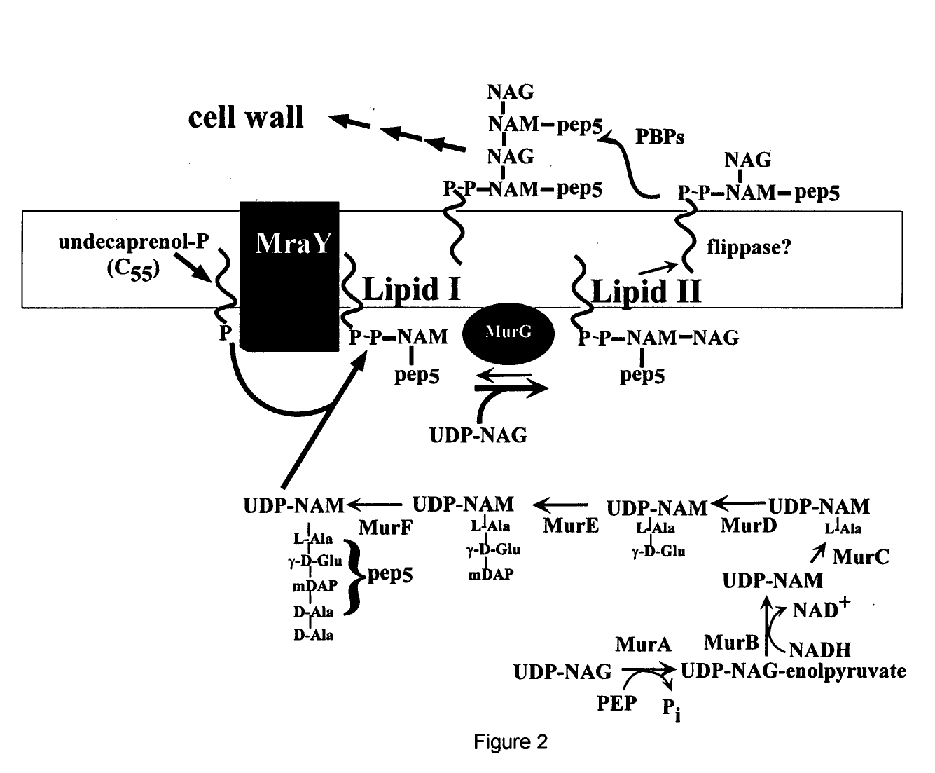

FIG. 3 shows the amino acid sequence of the E protein, with the pos mutations, and the basic structure of the E expression vector.

FIG. 4 shows a summary of the genetics of slyD and Epos.

FIG. 5 shows selection for eps host mutants resistant to Epos expression.

FIG. 6 shows that the eps phenotype is tightly linked to the 2 minute region (mra locus) of the E. coli chromosome.

FIG. 7 shows a recessive/dominant test for the eps mutation and Tn mapping strategy

FIG. 8 shows that the mraY mutation is responsible for the Eps phenotype in trans to the wt mraY.

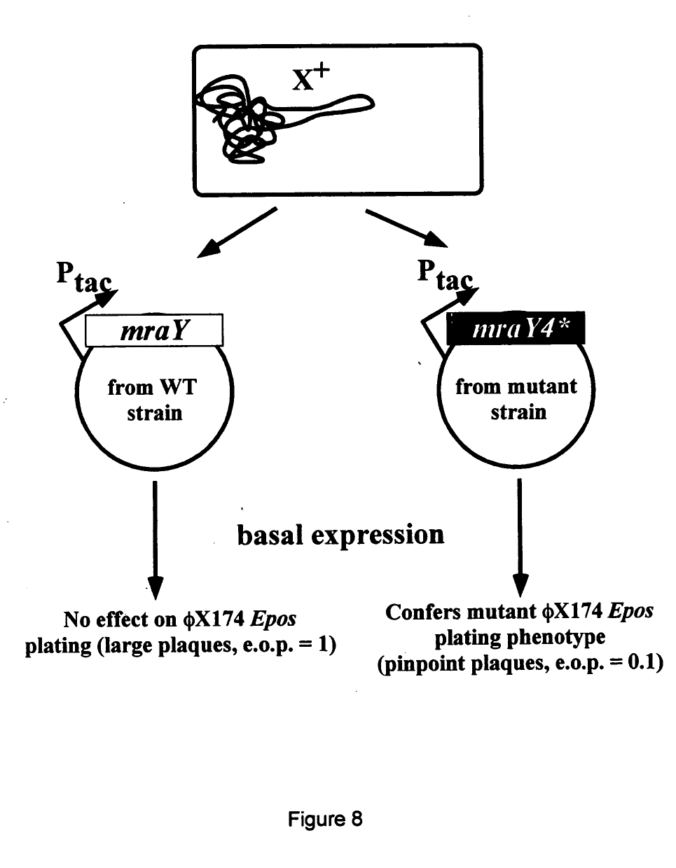

FIG. 9 shows the reaction catalyzed by MraY.

FIG. 10 shows lipid linked intermediates in bacterial cell wall synthesis.

FIG. 11 shows that MraY expressed from the araBAD promoter delays the onset of Emyc lysis, demonstrating that multicopy gene dosage can be used to screen for phages or lysis genes which target a cell wall synthesis gene.

FIG. 12 shows that MraY (F288L) expressed from the araBAD promoter inhibits Emyc lysis, demonstrating that multicopy dosage of a resistant cell wall enzyme gene can be used to screen for phages or lysis genes with altered and increased lytic function.

FIG. 13 shows a non-limiting model for a mechanism for E lysis and emphasizes that Epos acts in the same way but is present at a higher concentration than wt.

FIG. 14 shows that Epos protein is equally unstable as the E protein in a slyD mutant host. Pictured are gel analyses of pulse-labeled Emyc and Emycpos protein, immunoprecipitated by monoclonal antibody against the myc epitope, with varying chase periods in the absence of label.

FIG. 15 shows the method for selection of host rat mutants resistant to the A₂ expression and screening for resistance to the RNA phage Qβ and sensitivity to the RNA phage MS2.

FIG. 16 shows the MurA sequence and the position of the rat1 mutation.

FIG. 17 shows the 3-dimensional structure of MurA, as determined by crystallography, with the position of the rat1 mutation indicated.

FIG. 18 shows the steps in the pathway for making the bacterial cell wall which are inhibited by the phage single gene lysis proteins E and A₂.

FIG. 19 shows that in cells induced for E, incorporation of the labeled DAP is completely blocked before lysis.

FIG. 20 shows that in cells induced for A2, incorporation of the labeled DAP is completely blocked before lysis

FIG. 21 shows the DAP label accumulates in the pool of soluble, but not lipid-inked precursors or cell wall, in cells induced for E.

FIG. 22 shows the DAP label accumulates neither in the pool of soluble or lipid-inked precursors or cell wall, in cells induced for A₂.

FIG. 23 shows that MraY activity, as assessed by the exchange reaction, is inhibited in E-containing membranes as much as it is when the MraY inhibitor tunicamycin is present.

### DETAILED DESCRIPTION OF THE INVENTION

It is readily apparent to one skilled in the art that various embodiments and modifications may be made to the invention disclosed in this application without departing from the scope and spirit of the invention.

The term “bacteriophage” or “phage” as used herein is defined as a virus that infects bacteria. Phages, like other viruses, can be divided into those with RNA genomes e.g., mostly small and single stranded, those with small DNA genomes, e.g., generally less than 10 kb, mostly single stranded, and those with medium to large DNA genomes, e.g., 30-200 kb.

The term “cell wall” as used herein is defined as the peptidoglycan structure of eubacteria which gives shape and rigidity to the cell.

The term “envelope” as used herein is defined as the covering of bacteria which includes the cell wall, its connections to the outer membrane in Gram-negative bacteria, the outer membrane itself, including the lipopolysaccharide, and other outer components such flagella, pili, capsule and other proteins, such as M protein or S-layer proteins.

The term “Gram-negative bacteria” or “Gram-negative bacterium” as used herein is defined as bacteria which have been classified by the Gram stain as having a red stain. Gram-negative bacteria have thin walled cell membranes consisting of a single layer of peptidoglycan and an outer layer of lipopolysaccharide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary organisms not in the family Enterobacteriacea include, but are not limited to, Pseudomonas aeruginosa, Stenotrophomonas maltophilia, Burkholderia, Cepacia, Gardenerella, Vaginalis, and Acientobacter species.

The term “Gram-positive bacteria” or “Gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “peptidoglycan” as used herein is defined as a rigid mesh made up of ropelike linear polysaccharide chains cross-linked by peptides.

The term “polypeptide” as used herein is defined as a chain of amino acid residues, usually having a defined sequence. As used herein the term polypeptide is mutually inclusive of the terms “peptides” and “proteins”.

The term “polypeptide antibiotic” as used herein is defined as a protein or polypeptide produced from a single-gene lysis protein. Further, a skilled artisan recognizes that the protein or polypeptide antibiotic can be a fragment or a mutated lysis protein or lysis polypeptide. Also, contemplated is the use of random proteins that have been mutated to mimic the action of the polypeptide antibiotic.

The term “single-gene lysis polypeptide” as used herein is defined as the strategy in which a single-gene encodes a lysis protein that is involved in causing cell lysis. For example, small bacteriophages utilize the single-gene lysis polypeptide strategy. However, a skilled artisan recognizes that it is within the scope of the present invention that other non-phage sources, both biological and synthetic, may contain a single-gene lysis polypeptide. It is envisioned that the single-gene lysis polypeptide may be an evolutionary remnant of a phage which is captured by a host bacterium for its own purposes. For example, a bacterium may desire to lyse cells that are in a non-productive state, to eliminate replication errors during cell division or to eliminate cells that have been infected with a phage.

The term “target protein” or “target polypeptide” as used herein is defined as a protein or polypeptide that is involved in cell wall synthesis or synthesis of other envelope components essential for the integrity of the cell. A skilled artisan can recognize that this may also include any derivative or fragment thereof of the protein involved in cell wall synthesis or synthesis of other envelope components essential for the integrity of the cell.

I. Nucleic Acids

As discussed below, a “nucleic acid sequence” may contain a variety of different bases and yet still produce a corresponding polypeptide that is functionally indistinguishable.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the function of the target gene or lysis gene.

A. Nucleic Acids Encoding a Target Polypeptide or Lysis Polypeptide

Nucleic acids according to the present invention may encode an entire target polypeptide and/or single-gene lysis polypeptide, a domain of target polypeptide and/or lysis polypeptide, or any other fragment of the target polypeptide and/or lysis polypeptide as set forth herein. The nucleic acid may be derived from genomic DNA, i.e., cloned directly from the genome of a particular organism. Further, the nucleic acid may be derived from RNA. In preferred embodiments, however, the nucleic acid from RNA phages would comprise complementary DNA (cDNA).

The term “cDNA” is intended to refer to DNA prepared using messenger RNA (mRNA) as a template. Many of the viruses contain a RNA genome. It is contemplated to utilize these RNA genomes to screen for lysis polypeptides, thus, the RNA would be converted into DNA by standard methods of making “cDNA” from RNA.

It also is contemplated that a given protein from a given species may be represented by natural variants that have slightly different nucleic acid sequences but, nonetheless, encode the same protein (see Table 1 below).

TABLE 1

| | |
|---|---|
| Amino Acids | Codons |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp W | UGG | |
| Tyrosine | Tyr | Y | UAC UAU |

Allowing for the degeneracy of the genetic code, sequences that have at least 15 about 50%, usually at least about 60%, more usually about 70%, most usually about 80%, preferably at least about 90% and most preferably about 95% of nucleotides that are identical to the nucleotides of known sequences for bacterial target proteins and or lysis proteins are contemplated.

The DNA segments of the present invention include those encoding biologically functional equivalent bacterial target polypeptides and/or lysis polypeptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequences of the bacterial target gene or the lysis gene. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of a bacterial target gene or lysis gene under relatively stringent conditions such as those described herein. Such sequences may encode the entire protein or functional or non-functional fragments thereof.

Alternatively, the hybridizing segments may be shorter oligonucleotides. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 3431 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mm KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to the bacterial target protein or lysis protein or, more particularly, homologs of bacterial target protein or lysis protein from other species. Normally, the target DNA will be a genomic DNA library or a cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA'S, may be employed to inhibit gene transcription or translation or both within a host cell, either in vitro or in vivo.

Antisense constructs may be designed to bind to the promoter and other control regions of a gene. As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

C. Vectors for Cloning, Gene Transfer and Expression

Within certain embodiments expression vectors are employed to express a bacterial target polypeptide or a lysis polypeptide product, which can then be purified and, for example, be used to vaccinate animals to generate antisera or monoclonal antibody with which further studies may be conducted. Furthermore, it is within the scope of the present invention that the expression vectors may be used. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing cell clones expressing the products are also provided, as is an element that links expression of the drug selection markers to expression of the polypeptide.

(i) Regulatory Elements

Throughout this application, the term “expression construct” or “expression cassette” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box.

In the bacterial genome, there are several conserved features in a bacterial promoter: the start site or point, the 10-35 bp sequence upstream of the start site, and the distance between the 10-35 bp sequences upstream of the start site. The start point is usually (90% of the time) a purine. Upstream of the start site is a 6 bp region that is recognizable in most promoters. The distance varies from 9-18 bp upstream of the start site, however, the consensus sequence is TATAAT. Another conserved hexamer is centered at 35 bp upstream of the start site. This consensus sequence is TTGACA. Additional promoter elements regulate the frequency of transcriptional initiation. The spacing between promoter elements frequently is flexible, so that promoter function is preserved when elements are inverted or moved relative to one another.

In certain embodiments, viral promoters may be used. These promoters may be extremely primitive or complex depending upon the virus. For example, some viral promoters like the T4 phage promoter may only contain an AT-rich sequence at 10 bp upstream of the start site, but not a consensus sequence 35 bp upstream of the start site.

In certain embodiments, the lac promoter, T7 promoter, T3, SP6, or tac promoter can be used to obtain high-level expression of the coding sequence of interest. The use of other bacterial, viral or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Also contemplated is the use of the native promoter to drive the expression of the nucleic acid sequence. Further, selection of a promoter that is regulated in response to specific physiologic signals can permit inducible expression of the gene product, e.g. heat shock promoters.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et. al., 1988 and Ausubel et. al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

(iv) Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

(v) Expression Systems

Numerous expression systems exist that comprise at least a part or all of the compositions discussed above. Prokaryote-based systems can be employed for use with the present invention to produce nucleic acid sequences, or their cognate polypeptides, proteins and peptides. Many such systems are commercially and widely available.

One skilled in the art is aware of the various prokaryote-based expression systems. Exemplary systems from PROMEGA include, but are not limited to, pGEMEX®-1 vector, pGEMX®-2 Vector, and Pinpoint control Vectors. Examples from STRATAGENE® include, but are not limited to, pBK Phagemid Vector, which is inducible by IPTG, pSPUTK In vitro Translation Vector, pET Expression systems, Epicurian Coli® BL21 Competent Cells and pDual™ Expression System.

(vi) Delivery of Expression Vectors

In order to effect expression of sense or antisense gene constructs, the expression construct must be delivered into a cell. This delivery may be accomplished in vitro, as in laboratory procedures for transforming cells lines using well developed procedures. Transformation of bacterial cell lines can be achieved using a variety of techniques. One method includes using calcium chloride (Mandel and Higa, 1970). The exposure to the calcium ions renders the cells able to take up the DNA, or competent. Another method is electroporation. In this technique, a high-voltage electric field is applied briefly to cells, apparently producing transient holes in the cell membrane through which plasmid DNA enters (Shigekawa and Dower, 1988). These techniques and modifications are well known in the art. Thus, it is well within the scope of the present invention that a bacterial cell line may be transformed by any available transformation procedure or modification thereof.

II. Isolation of Peptides and Polypeptides

In addition to the entire molecule, the present invention also relates to fragments of the polypeptide that may or may not retain the various functions described below. Fragments, including the N-terminus of the molecule may be generated by genetic engineering of translation stop sites within the coding region (discussed below). Alternatively, treatment of the polypeptides with proteolytic enzymes, known as proteases, can produces a variety of N-terminal, C-terminal and internal fragments. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

A. Variants of Lysis and Target Polypeptides

Amino acid sequence variants of the polypeptide, e.g., lysis polypeptide and/or target polypeptide, can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of the lysis protein or target protein, but with altered and even improved characteristics.

B. Domain Switching

Domain switching involves the generation of chimeric molecules using different but, in this case, related polypeptides. By comparing various target or lysis proteins, one can make predictions as to the functionally significant regions of these molecules. It is possible, then, to switch related domains of these molecules in an effort to determine the criticality of these regions to target or lysis protein function. These molecules may have additional value in that these “chimeras” can be distinguished from natural molecules, while possibly providing the same function.

C. Fusion Proteins

A specialized kind of insertional variant is the fusion protein. This molecule generally has all or a substantial portion of the native molecule, linked at the N- or C-terminus, to all or a portion of a second polypeptide. For example, fusions typically employ leader sequences from other species to permit the recombinant expression of a protein in a heterologous host. Another useful fusion includes the addition of a immunologically active domain, such as an antibody epitope, to facilitate purification of the fusion protein. Inclusion of a cleavage site at or near the fusion junction will facilitate removal of the extraneous polypeptide after purification. Other useful fusions include linking of functional domains, such as active sites from enzymes, glycosylation domains, cellular targeting signals or transmembrane regions.

F. Purification of Proteins

It may be desirable to purify the target or lysis polypeptide or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et. al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

G. Synthetic Peptides

The present invention also includes smaller target or lysis-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young, (1984); Tam et. al., (1983); Merrifield, (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

H. Antigen Compositions

The present invention also provides for the use of target or lysis proteins or peptides as antigens for the immunization of animals relating to the production of antibodies. It is envisioned that the target or lysis polypeptide or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

III. Mutagenesis

Where employed, mutagenesis will be accomplished by a variety of standard, mutagenic procedures. Mutation is the process whereby changes occur in the quantity or structure of an organism. Mutation can involve modification of the nucleotide sequence of a single-gene, blocks of genes or whole chromosome. Changes in single-genes may be the consequence of point mutations which involve the removal, addition or substitution of a single nucleotide base within a DNA sequence, or they may be the consequence of changes involving the insertion or deletion of large numbers of nucleotides.

Mutations can arise spontaneously as a result of events such as errors in the fidelity of DNA replication or the movement of transposable genetic elements (transposons) within the genome. They also are induced following exposure to chemical or physical mutagens. Such mutation-inducing agents include ionizing radiations, ultraviolet light and a diverse array of chemical such as alkylating agents and polycyclic aromatic hydrocarbons all of which are capable of interacting either directly or indirectly (generally following some metabolic biotransformations) with nucleic acids. The DNA lesions induced by such environmental agents may lead to modifications of base sequence when the affected DNA is replicated or repaired and thus to a mutation. Mutation also can be site-directed through the use of particular targeting methods.

A. Random Mutagenesis

i) Spontaneous Mutagenesis

Spontaneous mutations occur in bacteria at a rate of approximately 10⁻⁵-10⁻⁶ events per locus per generation. In E. coli, the major cause of spontaneous mutation results from the presence of an unusual base in the DNA, e.g., modified bases. The most common modified base is 5-methylcytosine, which is generated by a methylase enzyme that adds a methyl group to a cytosine residue. This modified base provides a hotspot for spontaneous point mutations because it undergoes spontaneous deamination at a high frequency. Deamination results in the replacement of the amino group by a keto group converting 5-mehtylcytosine to thymine.

ii) Insertional Mutagenesis

Insertional mutagenesis is based on the inactivation of a gene via insertion of a known DNA fragment. Because it involves the insertion of some type of DNA fragment, the mutations generated are generally loss-of-function, rather than gain-of-function mutations. However, there are several examples of insertions generating gain-of-function mutations (Oppenheimer et. al., 1991). Insertion mutagenesis has been very successful in bacteria and Drosophila (Cooley et. al., 1988) and recently has become a powerful tool in corn (Schmidt et. al., 1987); Arabidopsis; (Marks et. al., 1991; Koncz et. al., 1990); and Antirrhinum (Sommer et. al., 1990).

Transposable genetic elements are DNA sequences that can move (transpose) from one place to another in the genome of a cell. The first transposable elements to be recognized were the Activator/Dissociation elements of Zea mays (McClintock, 1957). Since then, they have been identified in a wide range of organisms, both prokaryotic and eukaryotic.

Transposable elements in the genome are characterized by being flanked by direct repeats of a short sequence of DNA that has been duplicated during transposition and is called a target site duplication. Virtually all transposable elements whatever their type, and mechanism of transposition, make such duplications at the site of their insertion. In some cases the number of bases duplicated is constant, in other cases it may vary with each transposition event. Most transposable elements have inverted repeat sequences at their termini these terminal inverted repeats may be anything from a few bases to a few hundred bases long and in many cases they are known to be necessary for transposition.

Prokaryotic transposable elements have been most studied in E. coli and Gram negative bacteria, but also are present in Gram positive bacteria. They are generally termed insertion sequences if they are less than about 2 kB long, or transposons if they are longer. Bacteriophages such as mu and D108, which replicate by transposition, make up a third type of transposable element elements of each type encode at least one polypeptide a transposase, required for their own transposition. Transposons often further include genes coding for function unrelated to transposition, for example, antibiotic resistance genes.

Transposons can be divided into two classes according to their structure. First, compound or composite transposons have copies of an insertion sequence element at each end, usually in an inverted orientation. These transposons require transposases encoded by one of their terminal IS elements. The second class of transposon have terminal repeats of about 30 base pairs and do not contain sequences from IS elements.

Transposition usually is either conservative or replicative, although in some cases it can be both. In replicative transposition, one copy of the transposing element remains at the donor site, and another is inserted at the target site. In conservative transposition, the transposing element is excised from one site and inserted at another.

Eukaryotic elements also can be classified according to their structure and mechanism of transportation. The primary distinction is between elements that transpose via an RNA intermediate, and elements that transpose directly from DNA to DNA.

Elements that transpose via an RNA intermediate often are referred to as retrotransposons, and their most characteristic feature is that they encode polypeptides that are believed to have reverse transcriptionase activity. There are two types of retrotransposon. Some resemble the integrated proviral DNA of a retrovirus in that they have long direct repeat sequences, long terminal repeats (LTRs), at each end. The similarity between these retrotransposons and proviruses extends to their coding capacity. They contain sequences related to the gag and pol genes of a retrovirus, suggesting that they transpose by a mechanism related to a retroviral life cycle. Retrotransposons of the second type have no terminal repeats. They also code for gag- and pol-like polypeptides and transpose by reverse transcription of RNA intermediates, but do so by a mechanism that differs from that or retrovirus-like elements. Transposition by reverse transcription is a replicative process and does not require excision of an element from a donor site.

Transposable elements are an important source of spontaneous mutations, and have influenced the ways in which genes and genomes have evolved. They can inactivate genes by inserting within them, and can cause gross chromosomal rearrangements either directly, through the activity of their transposases, or indirectly, as a result of recombination between copies of an element scattered around the genome. Transposable elements that excise often do so imprecisely and may produce alleles coding for altered gene products if the number of bases added or deleted is a multiple of three.

Transposable elements themselves may evolve in unusual ways. If they were inherited like other DNA sequences, then copies of an element in one species would be more like copies in closely related species than copies in more distant species. This is not always the case, suggesting that transposable elements are occasionally transmitted horizontally from one species to another.

iii) Chemical mutagenesis

Chemical mutagenesis offers certain advantages, such as the ability to find a full range of mutant alleles with degrees of phenotypic severity, and is facile and inexpensive to perform. The majority of chemical carcinogens produce mutations in DNA. Benzo[a]pyrene, N-acetoxy-2-acetyl aminofluorene and aflotoxin B1 cause GC to TA transversions in bacteria and mammalian cells. Benzo[a]pyrene also can produce base substitutions such as AT to TA. N-nitroso compounds produce GC to AT transitions. Alkylation of the O4 position of thymine induced by exposure to n-nitrosoureas results in TA to CG transitions.

A high correlation between mutagenicity and carcinogenity is the underlying assumption behind the Ames test (McCann et. al., 1975) which speedily assays for mutants in a bacterial system, together with an added rat liver homogenate, which contains the microsomal cytochrome P450, to provide the metabolic activation of the mutagens where needed.

iv) Radiation Mutagenesis

The integrity of biological molecules is degraded by the ionizing radiation. Adsorption of the incident energy leads to the formation of ions and free radicals, and breakage of some covalent bonds. Susceptibility to radiation damage appears quite variable between molecules, and between different crystalline forms of the same molecule. It depends on the total accumulated dose, and also on the dose rate (as once free radicals are present, the molecular damage they cause depends on their natural diffusion rate and thus upon real time). Damage is reduced and controlled by making the sample as cold as possible.

Ionizing radiation causes DNA damage and cell killing, generally proportional to the dose rate. Ionizing radiation has been postulated to induce multiple biological effects by direct interaction with DNA, or through the formation of free radical species leading to DNA damage (Hall, 1988). These effects include gene mutations, malignant transformation, and cell killing. Ionizing radiation has been demonstrated to induce expression of certain DNA repair genes in some prokaryotic and lower eukaryotic cells (Borek, 1985).

In the present invention, the term “ionizing radiation” means radiation comprising particles or photons that have sufficient energy or can produce sufficient energy via nuclear interactions to produce ionization (gain or loss of electrons). An exemplary and preferred ionizing radiation is an x-radiation. The amount of ionizing radiation needed in a given cell generally depends upon the nature of that cell. Typically, an effective expression-inducing dose is less than a dose of ionizing radiation that causes cell damage or death directly. Means for determining an effective amount of radiation are well known in the art.

In a certain embodiments, an effective expression inducing amount is from about 2 to about 30 Gray (Gy) administered at a rate of from about 0.5 to about 2 Gy/minute. Even more preferably, an effective expression inducing amount of ionizing radiation is from about 5 to about 15 Gy. In other embodiments, doses of 2-9 Gy are used in single doses. An effective dose of ionizing radiation may be from 10 to 100 Gy, with 15 to 75 Gy being preferred, and 20 to 50 Gy being more preferred.

v) In vitro Scanning Mutagenesis

Random mutagenesis also may be introduced using error prone PCR (Cadwell and Joyce, 1992). The rate of mutagenesis may be increased by performing PCR in multiple tubes with dilutions of templates.

One particularly useful mutagenesis technique is alanine scanning mutagenesis in which a number of residues are substituted individually with the amino acid alanine so that the effects of losing side-chain interactions can be determined, while minimizing the risk of large-scale perturbations in protein conformation (Cunningham et. al., 1989).

In recent years, techniques for estimating the equilibrium constant for ligand binding using minuscule amounts of protein have been developed (Blackburn et. al., 1991; U.S. Pat. Nos. 5,221,605 and 5,238,808). The ability to perform functional assays with small amounts of material can be exploited to develop highly efficient, in vitro methodologies for the saturation mutagenesis of antibodies. The inventors bypassed cloning steps by combining PCR mutagenesis with coupled in vitro transcription/translation for the high throughput generation of protein mutants. Here, the PCR products are used directly as the template for the in vitro transcription/translation of the mutant single chain antibodies. Because of the high efficiency with which all 19 amino acid substitutions can be generated and analyzed in this way, it is now possible to perform saturation mutagenesis on numerous residues of interest, a process that can be described as in vitro scanning saturation mutagenesis (Burks et. al., 1997).

In vitro scanning saturation mutagenesis provides a rapid method for obtaining a large amount of structure-function information including: (i) identification of residues that modulate ligand binding specificity, (ii) a better understanding of ligand binding based on the identification of those amino acids that retain activity and those that abolish activity at a given location, (iii) an evaluation of the overall plasticity of an active site or protein subdomain, (iv) identification of amino acid substitutions that result in increased binding.

v) Random Mutagenesis by Fragmentation and Reassembly

A method for generating libraries of displayed polypeptides is described in U.S. Pat. No. 5,380,721. The method comprises obtaining polynucleotide library members, pooling and fragmenting the polynucleotides, and reforming fragments therefrom, performing PCR amplification, thereby homologously recombining the fragments to form a shuffled pool of recombined polynucleotides.

B. Site-Directed Mutagenesis

Structure-guided site-specific mutagenesis represents a powerful tool for the dissection and engineering of protein-ligand interactions (Wells, 1996, Braisted et. al., 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et. al., 1996, Brown et. al., 1996; Zeng et. al., 1996; Burton and Barbas, 1994; Yelton et. al., 1995; Jackson et. al., 1995; Short et. al., 1995; Wong et. al., 1996; Hilton et. al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

IV. Screening Assays

The present invention contemplates the screening of compounds for various abilities to interact and/or affect the production and/or function of cell wall synthesis or synthesis of other envelope components essential for the integrity of the cell or envelope development. Particularly preferred compounds will be those useful in inhibiting or promoting the peptidoglycan biosynthesis pathway. In the screening assays of the present invention, several different types of compounds will be screened for basic biochemical activity—e.g., binding to a target protein—and then tested for its ability to affect gene expression, protein production or protein function, at the cellular, tissue or whole animal level.

A. Modulators and Assay Formats

i) Assay Formats

The present invention provides methods of screening compounds, e.g., bacterial derived target polypeptides, lysis polypeptides or bacteriophages, for abilities to affect the production and/or function of cell wall or envelope development. In one embodiment, the present invention is directed to a method of:
(a) contacting bacteria with the lysis polypeptide;
(b) selecting for bacterial survivors of cell lysis caused by the lysis polypeptide that survive lysis by having a candidate bacterial nucleic acid sequence that encodes a target polypeptide making cells resistant to lysis; and
(d) mapping the candidate bacterial nucleic acid sequence, wherein the mapped sequence corresponds to the nucleic acid sequence which encodes the target polypeptide.

In yet another embodiment, the assay screens for candidate bacteriophages. The candidate bacteriophage can be isolated from sources selected from the group consisting of animal digestive tracts, fecal matter, sewage, waste water, natural salt water, fresh water and soil. Such methods would comprise, for example:
(a) obtaining a panel of recombinant bacterial strains, overexpressing at least one recombinant nucleic acid sequence encoding a target polypeptide involved in cell wall synthesis or synthesis of other envelope components essential for the integrity of the cell, or a non-target polypeptide as a control;
(b) obtaining a candidate bacteriophage;
(c) contacting the panel of recombinant bacterial strains with the candidate bacteriophage;
(d) selecting bacteriophage that is lysis-defective on at least one recombinant bacterial strain, wherein said bacteriophage expresses a single-gene lysis polypeptide that interacts with a target polypeptide involved in cell wall synthesis or synthesis of other envelope components essential for the integrity of the cell; and
(e) mapping a nucleic acid sequence in the bacteriophage, wherein the nucleic acid sequence encodes a single lysis polypeptide.

In yet another embodiment, the assay screens for candidate nucleic acid sequences that encode a single-gene lysis polypeptide. Such methods would comprise, for example:
(a) obtaining a library of DNA sequences cloned into an inducible plasmid expression vector;
(b) transforming the library into a bacterial strain

(c) contacting the bacterial strain with polypeptides produced from the library after induction;
(d) selecting for the vector plasmids that produce lysis polypeptides, wherein the vector plasmids are released into the medium after cell lysis; and
(e) determining the nucleic acid sequence encoding the lysis polypeptide from the plasmid DNA isolated from the lysed cells.

In yet another embodiment, the assay screens for candidate bacteriophages with enhanced lytic activity. Such methods would comprise, for example:
(a) obtaining a recombinant bacterial strain, wherein the bacterial strain is transformed with a vector comprising a nucleic acid sequence encoding a recombinant target polypeptide involved in cell wall synthesis or synthesis of other envelope components essential for the integrity of the cell;
(b) obtaining a candidate bacteriophage with a single-gene lysis polypeptide that interacts with the target polypeptide;
(c) contacting the recombinant bacterial strain with the candidate bacteriophage;
(d) selecting for survivor bacteriophages; and
(e) mapping the bacteriophage nucleic acid sequence which encodes the single-gene lysis polypeptide.

In still yet other embodiments, one would look at the effect of a single-gene lysis polypeptide or fragment or derivative thereof on the production of polypeptides involved in cell wall synthesis. This can be done by examining mRNA expression, although alterations in mRNA stability and translation would not be accounted for. A more direct way of assessing protein production is by directly examining protein levels, for example, through Western blot or ELISA. Other methods include, but are not limited to, the use of chromatography and mass spectrometry.

ii) Inhibitors and Activators

An inhibitor according to the present invention may be one which exerts an inhibitory affect on the production or function of a target polypeptide involved in cell wall synthesis or synthesis of other envelope components essential for the integrity of the cell. The inhibitor could be a bacteriophage-derived lysis protein, a protein modified to mimic the actions of a bacteriophage-derived lysis protein or a single-gene lysis protein from a non-phage source. Furthermore, an inhibitor includes any chemical compound that could be produced to mimic the action of a single-gene lysis protein. By the same token, an activator according to the present invention may be one which exerts a stimulatory effect on the production or function of a lysis protein resulting in an enhanced inhibitory effect on the production or function of a target polypeptide involved in cell wall synthesis.

iii) Candidate Substances

As used herein, the term “candidate substance” refers to any molecule that may potentially modulate or affect the expression or function of any polypeptide that is involved in cell wall synthesis or synthesis of other envelope components essential for the integrity of the cell. The candidate substance may be a protein or fragment thereof, a small molecule inhibitor, or even a nucleic acid molecule. It may prove to be the case that the most useful pharmacological compounds will be compounds that are structurally related to compounds which interact naturally with polypeptides involved in cell wall synthesis or synthesis of other envelope components essential for the integrity of the cell. Creating and examining the action of such molecules is known as “rational drug design,” and include making predictions relating to the structure of the target molecules (polypeptides involved in cell wall synthesis or synthesis of other envelope components essential for the integrity of the cell) and the candidate substance.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like the polypeptides involved in cell wall synthesis or synthesis of other envelope components essential for the integrity of the cell, and then design a molecule for its ability to interact with these polypeptides. Alternatively, one could design a partially functional fragment of these polypeptides (binding, but no activity), thereby creating a competitive inhibitor. This could be accomplished by x-ray crystallography, computer modeling or by a combination of both approaches. Another alternative would be to design a molecule similar to the single-gene lysis polypeptides (either bacteriophage-derived or non-phage derived).

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

Other suitable inhibitors include antisense molecules and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

B. In vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds (lysis polypeptides or derivatives thereof) that bind to a target polypeptide involved in cell wall synthesis or synthesis of other envelope components essential for the integrity of the cell or fragment thereof is provided.

The target polypeptide may be either free in solution, fixed to a support, expressed in or on the surface of a cell. Either the target polypeptide or the candidate compound (lysis polypeptide) may be labeled, thereby permitting determining of binding. In another embodiment, the assay may measure the inhibition of binding of a target polypeptide to a natural or artificial substrate or binding partner. Competitive binding assays can be performed in which one of the agents (a target polypeptide, for example) is labeled. Usually, the target polypeptide will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a cell synthesis target proteins and washed. Bound polypeptide is detected by various methods.

Purified target polypeptides can be coated directly onto plates for use in the aforementioned drug screening techniques. However, non-neutralizing antibodies to the polypeptides can be used to immobilize the proteins to a solid phase. Also, fusion proteins containing a reactive region (preferably a terminal region) may be used to link an active region (e.g., the C-terminus of the polypeptide to a solid phase.

C. In Cyto Assays

Various cell lines that overexpress the bacterial target polypeptides can be utilized for screening of candidate single-gene lysis substances. For example, cells containing a bacterial target protein with an engineered indicator can be used to study various functional attributes of candidate compounds. In such assays, the compound would be formulated appropriately, given its biochemical nature, and contacted with a target cell. Also contemplated is the use of various cell lines that express a candidate single-gene lysis polypeptide upon induction of the plasmid expression vector. These transformed cell lines can be utilized for screening of candidate target polypeptides.

Depending on the assay, culture may be required. As discussed above, the cell may then be examined by virtue of a number of different physiologic assays (growth or size). Alternatively, molecular analysis may be performed in which the function of bacterial target protein and related pathways may be explored. This involves assays such as those for protein expression, enzyme function, substrate utilization, mRNA expression and others.

D. In vivo Assays

The present invention particularly contemplates the use of various animal models. Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

V. Drug Formulations and Routes for Administration to Patients

Where clinical applications are contemplated, it will be necessary to prepare pharmaceutical compositions—expression vectors, virus stocks and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

### EXAMPLE 1

### DNA Cloning, PCR and Sequencing Methods; Bacterial Strains, Plasmids and Growth Conditions

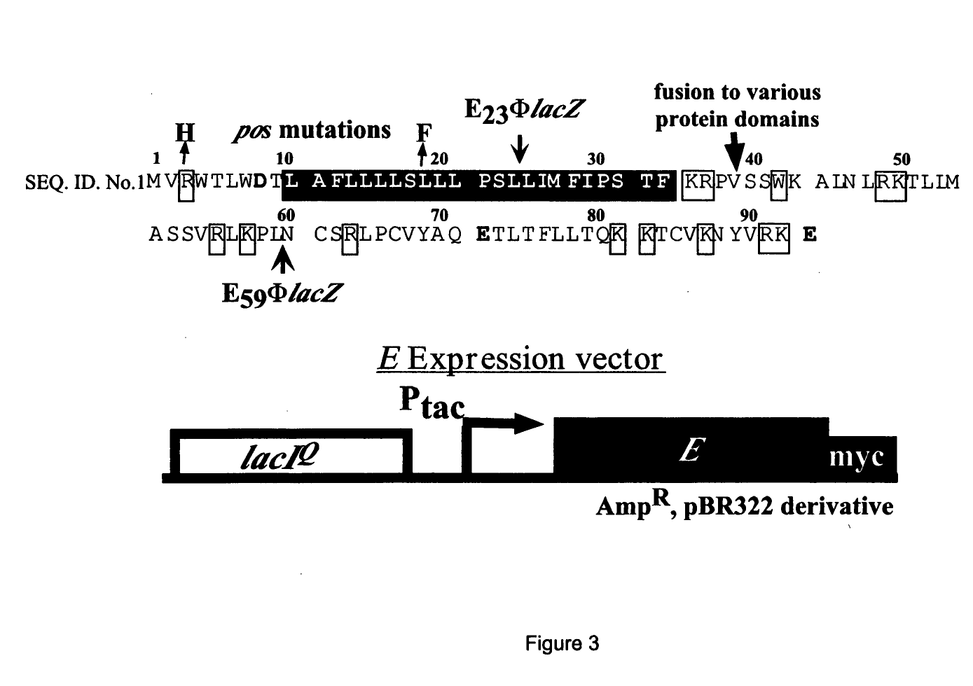

All DNA manipulations, including PCR, were performed according to standard and published procedures (Maniatis et. al., 1982 and Smith et. al., 1998) except as detailed in Bernhardt et. al., (2000). φX174Epos4B, referred to as φX174Epos, was isolated as a spontaneous plaque former on a slyD mutant lawn (W. D. Roof., unpublished results). Most plasmids and strains have been described (Bernhardt, et al., 2000). The Epos4B allele contains both the R3H and L19F missense mutations and henceforth will be referred to as Epos. E. coli K-12 strain ET505 (W3110 lysA::Tn10) was the host strain used in the work on MraY inhibition and was obtained from the E. coli Genetic Stock Center (New Haven, Conn.) (www.cgsg.biology.yale.edu). A lysA strain was required to prevent the conversion of added [³H]-DAP to Lys, so that [³H]-DAP can only be incorporated into cell wall and its precursors. The plasmid pEmycZK, described previously (Bernhart et. al., 2000) contains Emyc, encoding E with a C-terminal c-myc epitope tag, cloned under control of the IPTG-inducible tac promoter (FIG. 3). The control vector pJFlacZK is isogenic to pEmycZK except that it does not contain Emyc. It was constructed by inserting the lacZ gene in the HindIII site of pJF118EH (Fürste, Pansegrau, et. al., 1986) and converting it to KanR as described previously for pEmycZK (Bernhardt, et. al., 2000). Microbiological methods, culture growth conditions, phage plating and lysis profiles have been described previously (Bernhardt, et. al., 2000 and Roof et. al., 1994).

### EXAMPLE 2

### Genetic Techniques, Transposon Mutagenesis and Methods of Mutant Selection

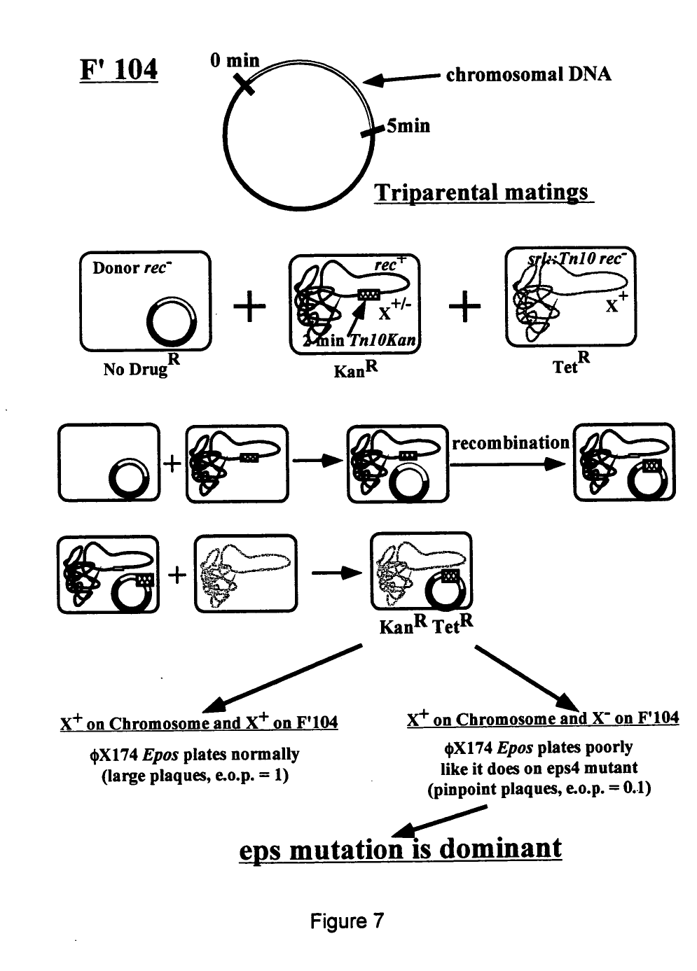

Standard bacterial matings were performed essentially as described (Miller, 1992). Triparental matings to generate a merodiploid with eps+ on the chromosome and eps4 on F′104 were performed by mixing 0.5 ml of exponential cultures of KL723 (strain 1), RY7283 (strain 2), and RY7278 (strain 3) and allowing them to stand at 37° C. for 5 h. The desired exconjugants were selected by plating dilutions on LB-Kan-tetracycline (FIG. 7). To generate homozygous eps+ merodiploids RY7281 was used as strain 2. P1 transductions were performed essentially as described (Miller, 1992). φX174Epos phage plating was performed as described (Roof, et. al., 1997).

MiniTncam transposon mutagenesis was performed on strain RY7285 (the exconjugant selected from triparental matings) by using the delivery phage NK1324 essentially as described (Kleckner, et. al., 1991) except the transposition mixture contained 0.5 ml of a 30× concentrated exponential culture of RY7285 in LB-IPTG-10 mM MgSO₄. Transposon insertions in the F′ were isolated by mating the pool of transposon mutants with RY2788 and selecting on LB-rifampicin-Kan-Cam. Insertions that eliminated the phage-resistant phenotype conferred by the eps4 allele on the F′ were identified by replica plating for φX174Epos sensitivity. Replica plating was performed by replicating plates containing about 200 colonies on velvet to plates with and without 10⁸ plaque-forming units of φX174Epos.

To select for Epos-resistant mutants, a culture of CCX1 pKN104B was grown to an A₅₅₀ of 0.18, and Epos expression was induced with IPTG. After lysis was complete (approximately 3.5 h), 0.1 ml of the culture was plated on LB-Kan-IPTG to yield approximately 200 colonies per plate. A total of about 2,000 survivors were isolated and screened for φX174Epos phage resistance by using cross-streaks. For cross-streaks, approximately 10⁷ plaque-forming units were spread down the center of a plate and allowed to dry. Survivor colonies were picked directly from the selection plate and streaked across the spread phage. A streak was scored positive if there was significant and reproducible growth across the phage.

To select for A₂-resistant mutants, the same procedure was followed except the culture was the male strain XL1-Blue pGL101 (Winter and Gold, 1983). In this protocol, these cells were induced with 1 mM IPTG in mid-logarithmic phase LB-Amp culture. After culture lysis, the surviving cells were concentrated and plated on LB-Amp agar and incubated overnight at 37° C. Approximately 10⁻⁶ of the original viable cell count survived the induction and plating. Colonies were screened by cross-streaking with the RNA phages Qβ and MS2.

### EXAMPLE 3

### Methods of Cell Wall Labeling and Precursor Analysis

Cell wall synthesis was measured as SDS-insoluble incorporation as follows. For E, ET505 pEmycZK and ET505 pJFlacZK were grown in minimal M9 glucose media in a 250 mL culture flasks at 37° C. to an A₅₅₀ of approximately 0.3 when a portion of each culture was transferred to a small pre-warned 50 mL flask containing sufficient [³H]-DAP to give a final activity of 5 microC/mL. For A₂, ET505 pA₂ and ET505 pJFlacZK were used. Constant aeration of all cultures was maintained throughout the experiment. After a 10 min pre-labeling period, both labeled and unlabeled cultures were induced with IPTG. Culture growth was monitored from the unlabeled culture and [³H]-DAP incorporation into cell wall was monitored in the labeled culture as described previously (Wientjes, et. al., 1985) with minor modifications. Briefly, 0.2 mL aliquots were removed at the indicated times and added directly into 0.8 mL of boiling 5% SDS. The samples were boiled for 1 hr and allowed to cool to RT before filtering through a 0.22 μm mixed cellulose-ester filter (type-GS, Millipore, Bedford, Mass.). The filters were washed with 30 mL distilled H₂O, allowed to dry completely, and the radioactivity associated with the cell wall was determined by counting the filters in a Beckman LS5000TD liquid scintillation counter using EcoscintA liquid scintillation fluid (National Diagnostics, Atlanta, Ga.). In control experiments, label incorporation into the cell wall was linear 10 min after addition of [³H]-DAP, indicating that the precursor pools were in isotopic equilibrium (data not shown).

Cell wall precursors were analyzed as follows. Cultures were grown as described above except to an A₅₅₀ of 0.6 and were induced with IPTG. After 2 min, a portion of the culture was added to a pre-warmed 50 mL flask containing sufficient [³H]-DAP for a total activity of 35 μCi/mL. Constant aeration of all cultures was maintained throughout the experiment. After an 8 min pulse-labeling period, prior to any observable lysis, three 1 mL aliquots of labeled culture were removed and centrifuged for 10 min at 4° C. at maximum speed in a microcentrifuge. The cell pellets were washed with 1 mL of ice-cold media and resuspended in 10 μL of dH₂O. The cell suspension was spotted on Whatman 3MM paper and labeled cell wall precursors were separated by development with solvent system A for approximately 20 hr as described (Lugtenberg & Haan 1971). Each lane was cut into 1 cm strips and counted as described above. Cell wall, nucleotide, and lipid intermediate fractions ran at published Rf values (0, 0. 1, and 0.8 respectively).

### EXAMPLE 4

### Selection for Epos Mutants

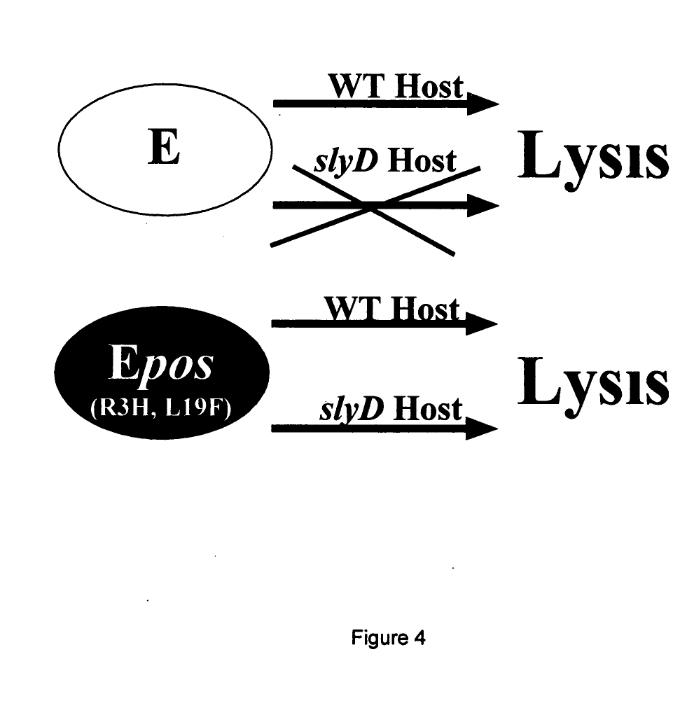

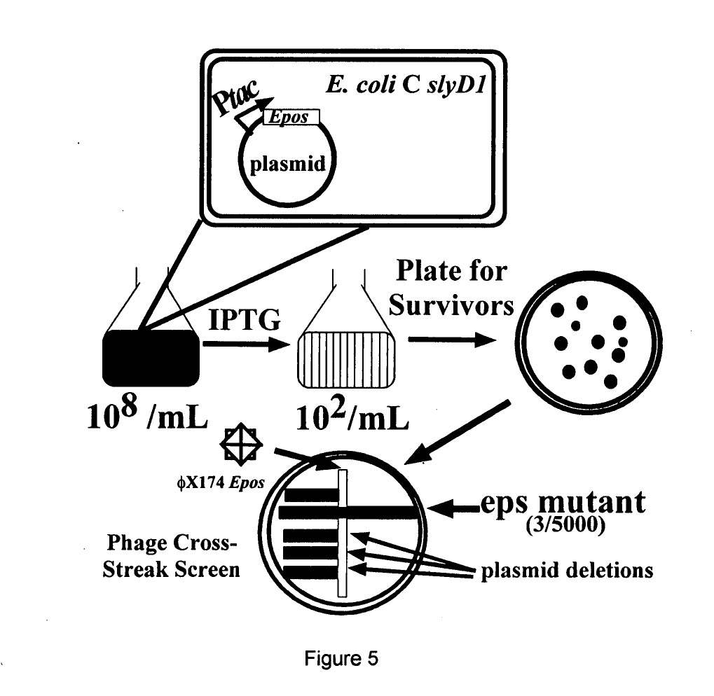

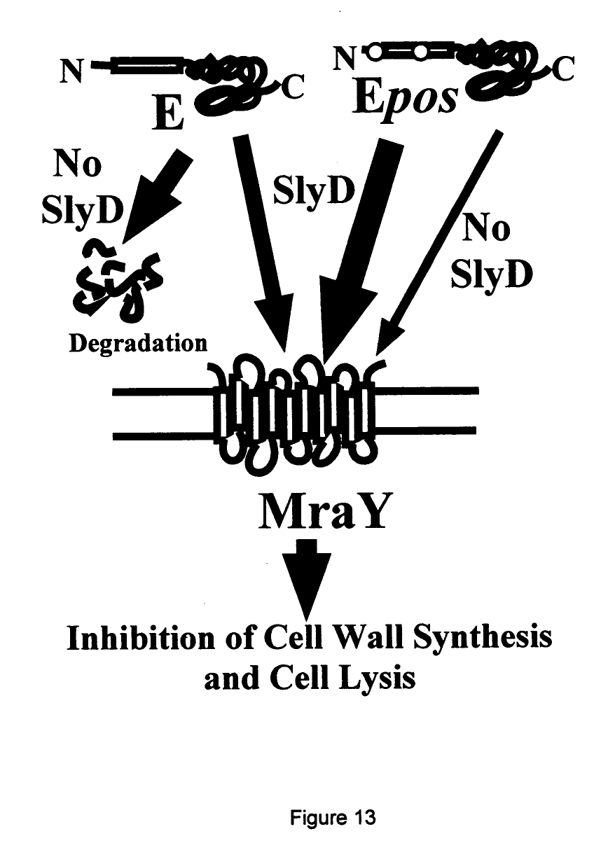

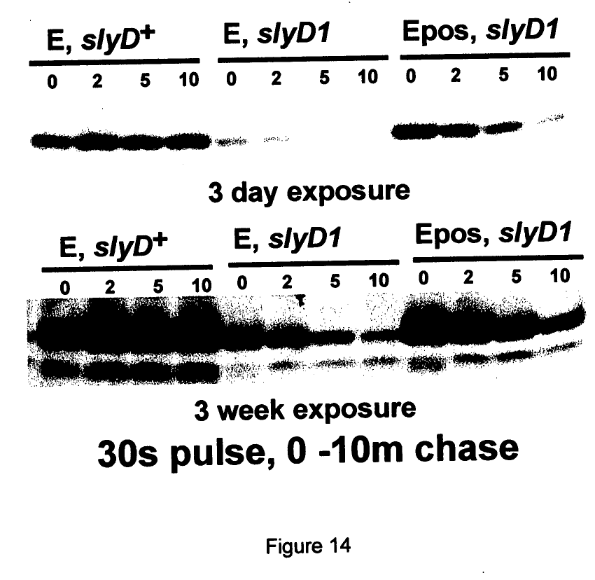

Epos (plates on slyD) mutants were originally isolated by selecting for φX174 plaques on a slyD mutant lawn (FIG. 3 and FIG. 4) (See Example 1). Since this original selection, numerous selections, employing both phage and plasmid based systems, were isolated with the same two missense mutations, R3H and L19F (FIG. 3). These same changes, among others, are naturally occurring in the related G4 phage E protein, which also lacks a slyD requirement for lysis. The double missense mutant, Epos4B, was also isolated and displays better lysis characteristics on a slyD mutant host than either of the single missense alleles. The Epos protein is equally unstable as the E protein in a slyD mutant host but it is synthesized at a much higher level (FIG. 14). This explains why not only are Epos mutants functional in a slyD mutant but in fact because of the higher expression levels the mutants exceed the lysis proficiency of E⁺ in a wt host. The existence of these mutations in E allowing bypass of the slyD requirement for lysis by higher expression levels demonstrate that slyD is serving an ancillary role in lysis and is not required for the lysis mechanism and also that there is no fundamental difference in the way that Epos and E proteins cause lysis (FIG. 13). This discovery leads to the strategy of using the lethal capacity of the Epos allele to select for mutations in the target gene of the host (FIG. 5).

### EXAMPLE 5

### Selection and Screen for Epos-Resistant Mutants

To identify the gene encoding the target of E lysis, spontaneous mutants of CCX1, E. coli C slyD1, that gain resistance to Epos expression from the plasmid pKN104B were selected (See Example 2) (FIG. 5). The majority of the selected survivors contained plasmid mutations that eliminated Epos expression. To identify host mutants conferring resistance to Epos in the high background of plasmid mutants, the survivors were screened by cross-streaking them against the φX174Epos phage. Survivors resulting from plasmid mutations are still sensitive to lysis by the phage-encoded Epos and were thus phage sensitive. True Epos-resistant host mutants were expected to be resistant to Epos from the phage as well as the plasmid. Approximately 2,000 survivors were screened, and 17 eps (Epos sensitivity) mutants scored positive for phage resistance. Two types of eps mutants were isolated, 14 with a partial phage-resistance phenotype and three with a tight resistance phenotype.

### EXAMPLE 6

### Mapping the Epos-Resistance Mutations to mraY

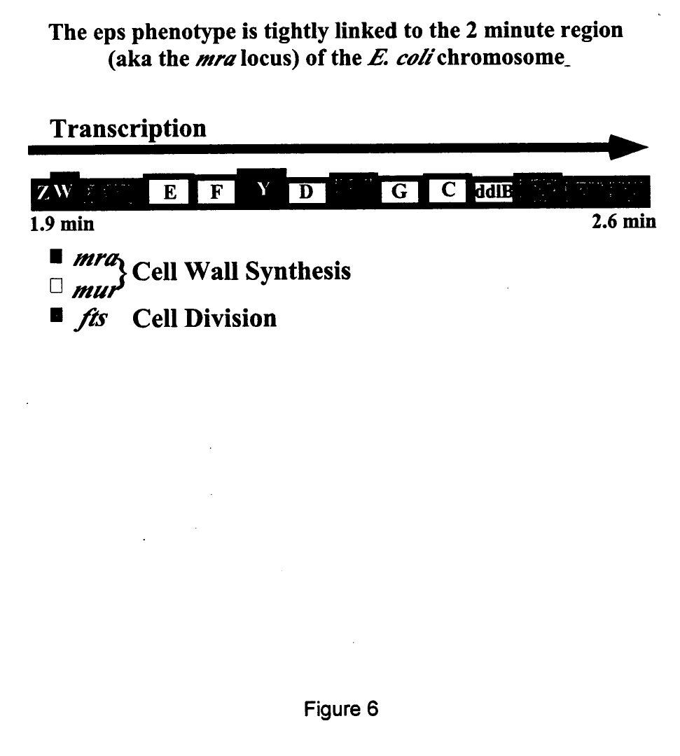

Hfr and P1 mapping localized the eps mutations to the 2 minute region of the E. coli chromosome (60% cotransducible with a Tn10 marker at 2 minutes). The 2 minute region contains the mra locus which is rich in genes for cell wall synthesis and cell division (FIG. 6) (Hara, et al., 1997 and Mengin-Lecreulx, et al., 1998). To assess the recessive or dominance of the eps4 allele, a tri-parental mating of F′ 104 was used to generate merodiploids of the 0-5 minute region of the chromosome (FIG. 7). Merodiploids containing two wt copies of the 0-5 minute region showed normal φX174 Epos plating efficiency and plaque size. However, merodiploids containing an eps⁺ allele on the chromosome and an eps4 allele on F′104 had the phage-resistant phenotype. Therefore, the eps4 allele is dominant over wt (FIG. 7).

To identify the gene containing the dominant eps4 mutation, a strain carrying F′104eps4 was mutagenized with mini-Tncam and mated with an eps⁺ strain (See Example 2). Exconjugants, carrying transposon insertions in the F′, were screened for φX174Epos sensitivity by replica plating. The positions of mini-Tncam insertions that eliminated the phage-resistance phenotype associated with the F′ were determined by inverse PCR and sequencing. Two insertions mapping to ftsI and murE were obtained, both with partial phage-sensitivity, suggesting the insertions were polar on the eps locus rather than knockouts. Knowing that the lytic function of E is contained in its hydrophobic membrane domain, the inventors reasoned that its cellular target should be a membrane protein. The first gene downstream of the transposon insertions encoding a membrane protein is mraY. The inventors amplified the mraY alleles from the parental and mutant strains and inserted them under control of the tac promoter in the vector pJF118 (Furste, et. al., 1986). As shown in Table 2, basal expression of mraY cloned from the mutant strains (pmraY4 and pmraY39) conferred the φX174Epos plating defect to the parental strain CCX1. On the other hand, basal expression of mraY cloned from the parental strain (pmraY) had only a slight phage-plating defect. Therefore, dominant mutations in mraY, a gene encoding a membrane bound enzyme involved in cell wall synthesis, confer the Eps phenotype. The eps alleles have been renamed as mraY4, mraY15, and mraY39.

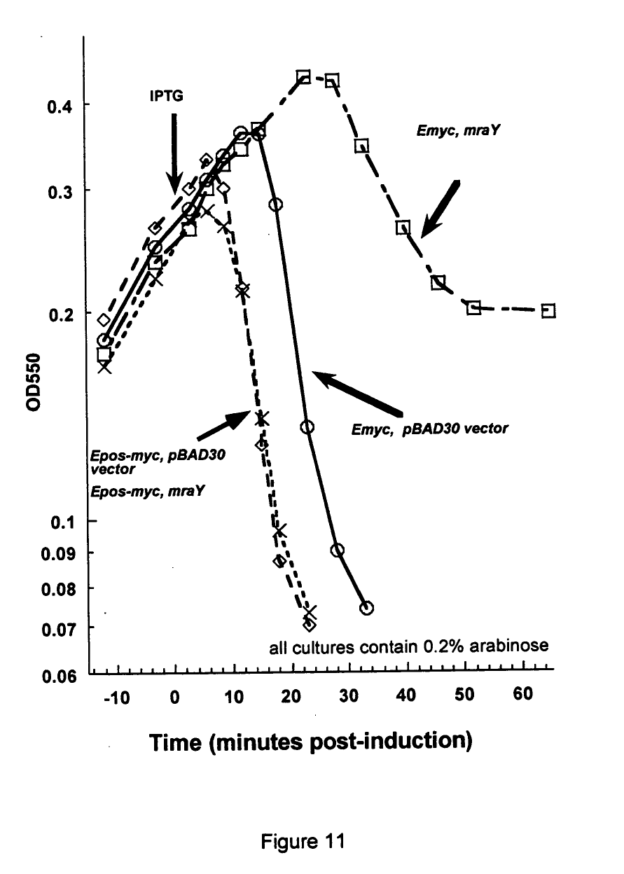

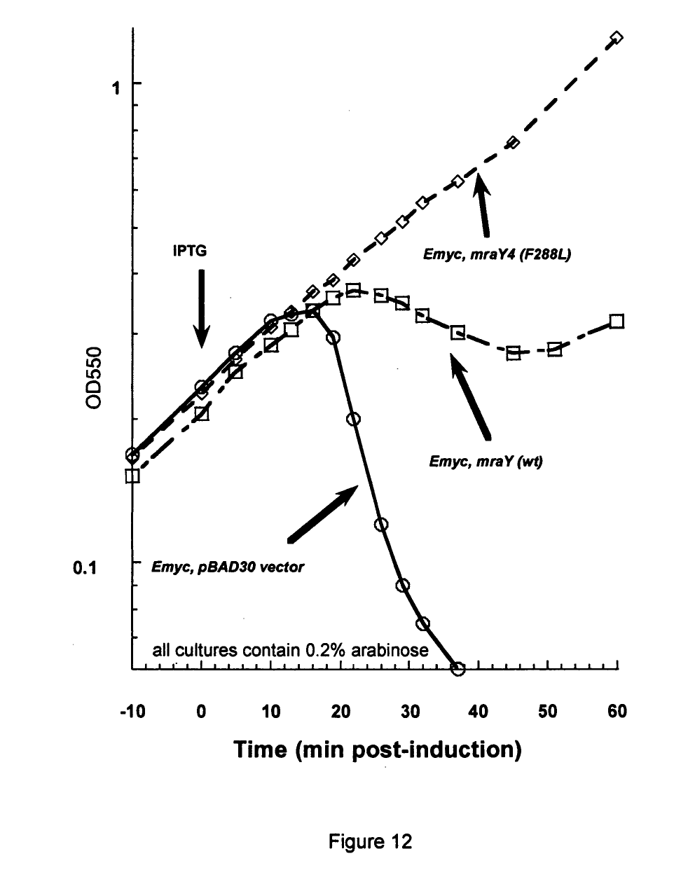

Two independent mutant alleles were sequenced and found to encode alterations in the primary structure of MraY: MraY4 has the change F288L and MraY15 has the change ΔL172. The presence of these single mutations in these two spontaneous mutants is genetic proof that MraY is the target of E. Moreover, multicopy plasmids carrying wild-type (E-sensitive) mraY require a much longer expression period for the E gene before lysis is detected (FIG. 11). Also, expression of the eps4 allele of mraY, resistant to Epos, from a multicopy plasmid, blocks E lytic function, even with the wt, E-sensitive allele on the chromosome (FIG. 12). These data indicate that E inhibits MraY on a stoichiometric basis and suggests that E binds MraY as part of its inhibitory function (FIG. 13).

Table 2 shows that MraY activity, but not the activity of the related enzyme Rfe, is inhibited in E-containing membranes, illustrating that E is a specific inhibitor of MraY.

TABLE 2
MraY and Rfe exchange reactions

| | cpm | |
|---|---|---|
| | UDP-MurNAc- | |
| membranes | pentapeptide^{a} | UDP-GlcNAc^{b} |
| ET505 pJFlacZK | 16100 ± 500 | 21700 ± 1400 |
| ET505 pEmycZK | 4000 ± 100 | 26800 ± 1800 |
| ET505 pJFlacZK + tunicamycin | 2000 | 3400 ± 500 |

^{a}cpm of [³H]-UMP converted to [³H]-UDP-MurNAc-pentapeptide in membrane preparations by MraY. The results are the average of 3 experiments ± the standard deviation. Tunicamycin results are the average of duplicate experiments.
^{b}cpm of [³H]-UMP converted to [³H]-UDP-GlcNAc in membrane preparations by Rfe. The results are the average of 3 experiments ± the standard deviation.

### EXAMPLE 7

### Host MraY is Inhibited by E

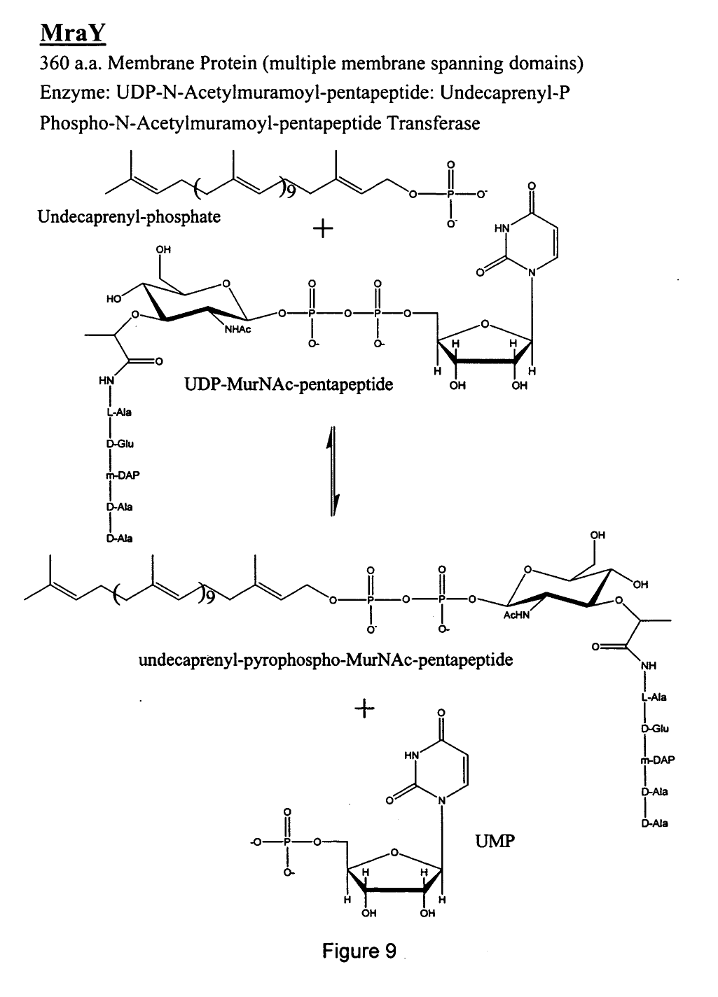

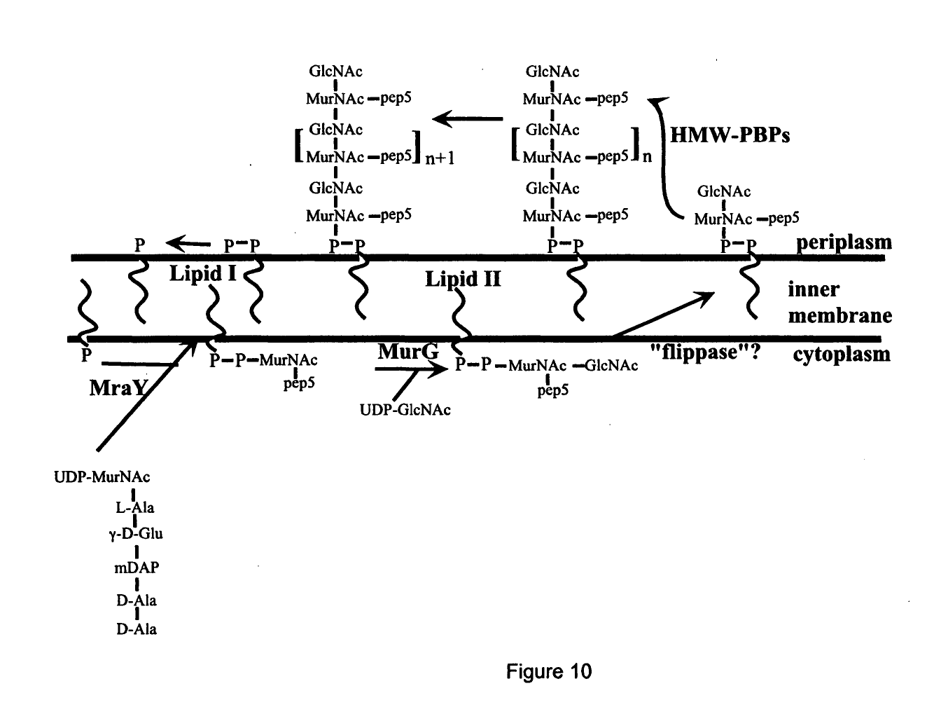

The results of these experiments, shown schematically in FIG. 9, FIG. 10 and FIG. 13, show that classical bacterial virus φX174 encodes a single small membrane protein E that causes cell lysis by inhibiting host MraY (a phospho-N-acetylmuramyl-pentapeptide-translocase; FIG. 9), an enzyme that catalyzes a crucial step in the pathway for making the cell wall (FIG. 10). While not being limited by theory, it is believed that when MraY activity is inhibited, for example by E (FIG. 13), the host cell attempts to divide but essentially blows up, or lyses, because it fails to make the new cell wall, or septum, which defines the new daughter cells. However, the inhibition of cell wall synthesis is generally lethal to bacteria as long as growth occurs, and thus the lethal and lytic effect of these cell wall synthesis inhibitors is not exclusively limited to the septal region.

### EXAMPLE 8

### Selection and Mapping of Rat Mutants (Resistant to A₂)

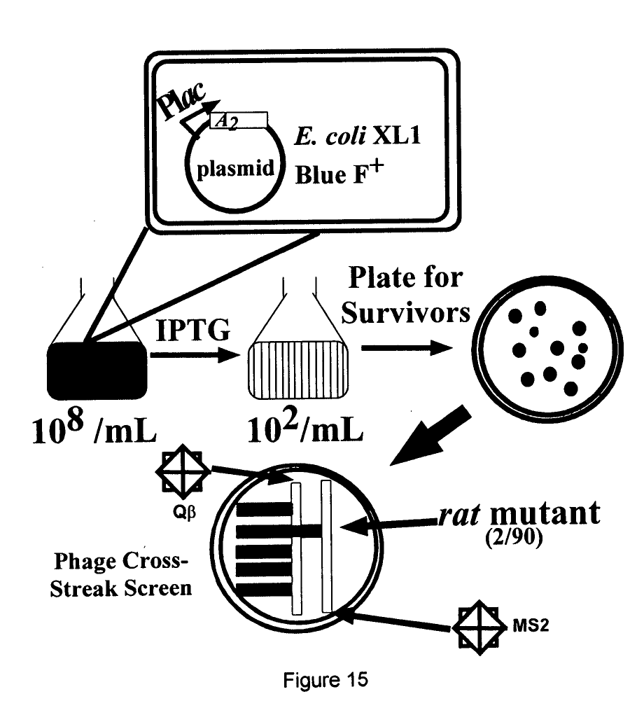

The male-specific RNA bacteriophage Qβ A₂ gene causes lysis when expressed from the phage or a bacterial plasmid. Therefore, genetic selections for host mutants resistant to A₂ expression were performed, with the goal of identifying the target of the A₂ protein (See Example 2). The colonies arising were considered candidates for the Rat phenotype (resistance to A-two). Ninety mutants selected which were in this way were screened for resistance to Qβ phage and sensitivity to the RNA phage MS2, which uses a different single-gene lysis system. Two survivors passing this screen (Qβ^{R}, MS2^{S}) were chosen and designated rat1 and rat2 (FIG. 15).

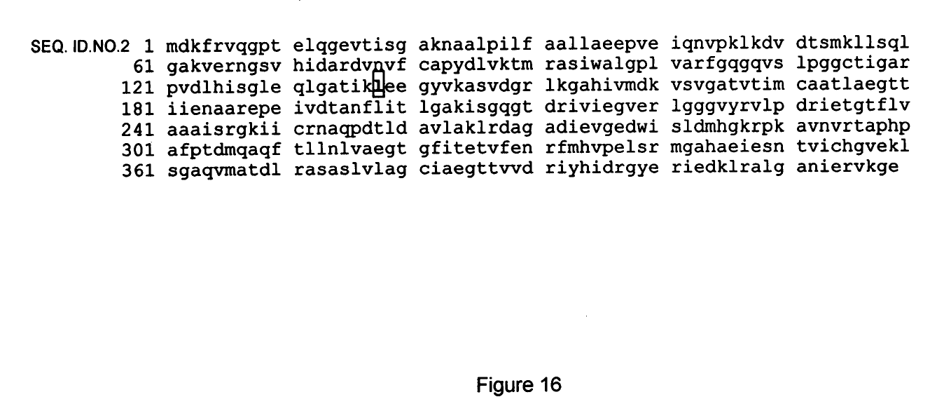

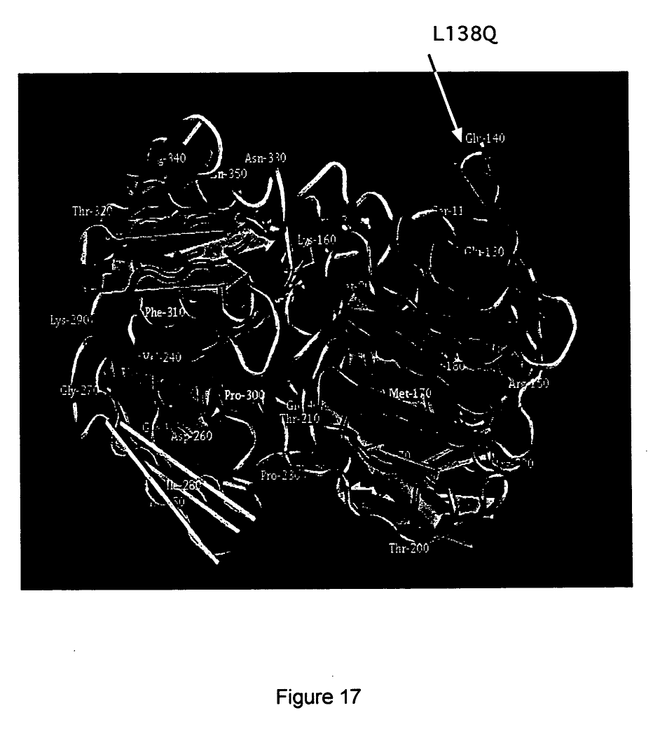

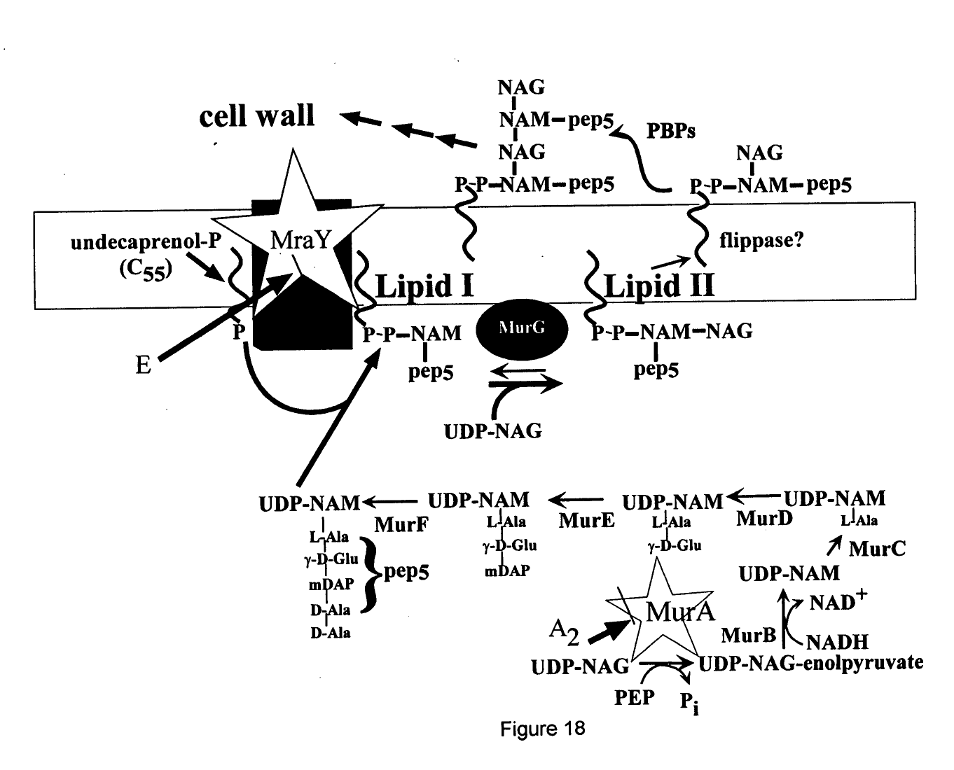

Genetic analysis was done on these mutants. The first locus checked is the cluster of cell-wall synthesis genes at 2 min on the chromosome, which includes mraY, the target of E. However, P1 transduction using a 2 min transposon marker revealed that 0 of 31 transductants lost the Rat phenotype, indicating that rat was not located in this cluster. Biochemical analysis of the cell wall precursor pools (see below) revealed that not only is cell wall biosynthesis blocked but also that no soluble UDP-MurNac-pentapeptide was present in the cells in which the A₂ was induced. The combination of these results narrowed the possible cell wall biosynthesis genes which might be the locus for rat mutations to murA, murB and murC. P1 transduction with a transposon insertion linked to murA revealed high linkage of the rat phenotype to the transposon. The murA genes from rat1 and rat2 were amplified by PCR and sequenced. A mutation was found, identical in each mutant, which converted Leu138 to Gln (FIG. 16). The altered amino acid residue occupies a position that controls access to the catalytic cleft of the MurA enzyme (FIG. 17). Thus rat1 and rat2 were siblings and allelic to murA. Because these were spontaneous mutations, unassociated with any mutagenesis, the finding of this mutation in the sequence of murA, combined with the blockage of the synthesis of the soluble precursor pool, is proof that the target of A₂ is MurA. Thus the results of these experiments, shown schematically in FIG. 15, FIG. 16 and FIG. 18 show that classical bacterial virus Qβ encodes a single protein A₂ that causes cell lysis by inhibiting host MurA (a UDP-NAG carboxyvinyltransferase), an enzyme that catalyzes a crucial step in the pathway for making the cell wall (FIG. 18). While not being limited by theory, it is believed that when MurA activity is inhibited, for example by A₂, the host cell attempts to divide but essentially blows up, or lyses, because it does not make the new cell wall necessary to separate viable daughter cells.

### EXAMPLE 9

### Biochemical Analysis of E and A₂ Function

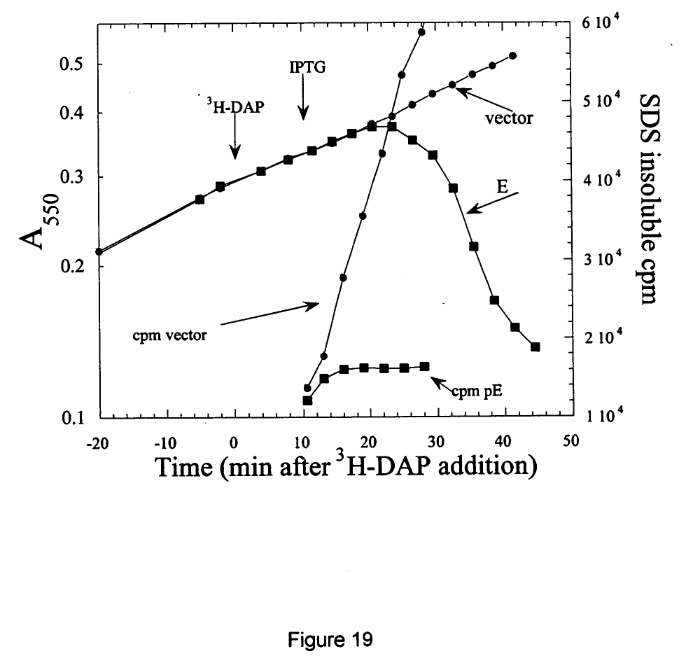

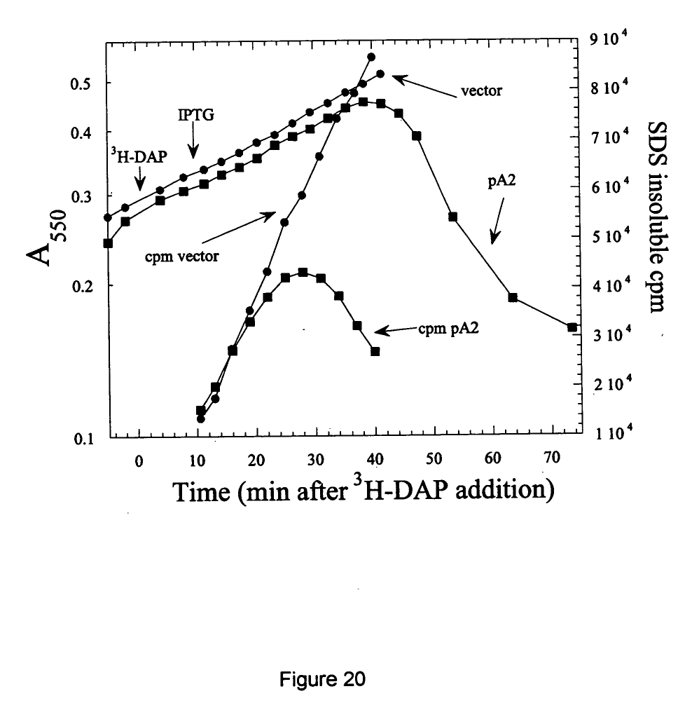

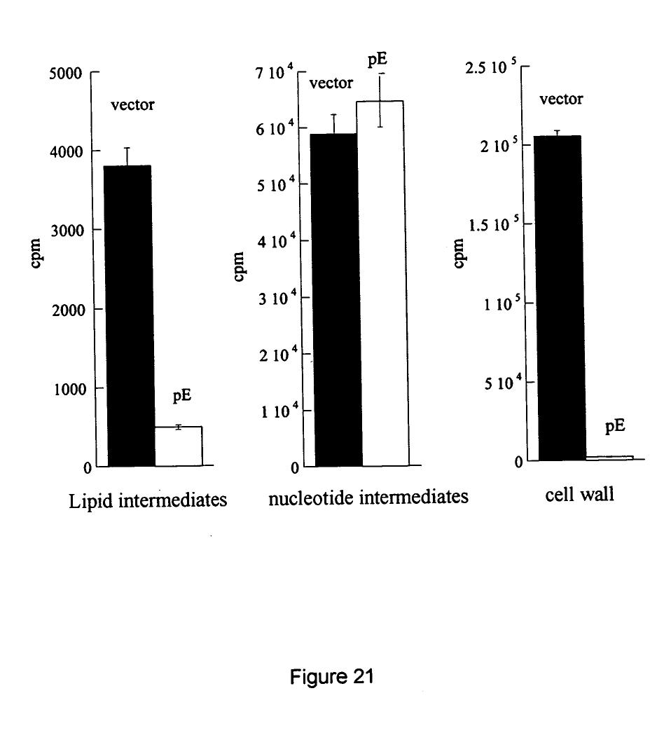

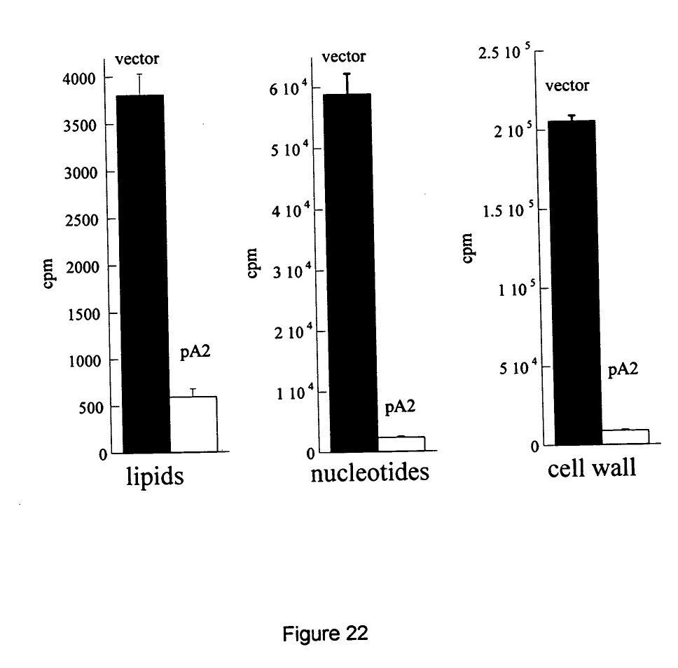

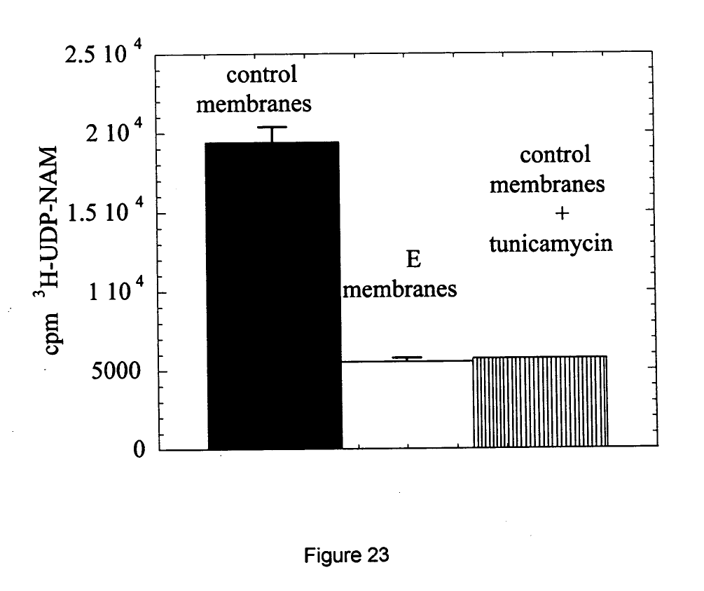

Biochemical investigation has confirmed the above genetic identifications. In both systems, the effect of the lysis gene induction on peptidoglycan synthesis was assessed by labeling with radioactive diaminopimelic acid, a component of the pentapeptide moeity unique to peptidoglycan. It was observed that in both cases, the incorporation of label was completely blocked long before lysis was detected (FIG. 19 and FIG. 20). Peptidoglycan precursors were analyzed in both systems. After induction of E or A₂, no undecaprenol-linked precursor was detected, indicating the block was at MraY or earlier in the pathway (FIG. 21 and FIG. 22). In cells expressing E, there was accumulation of a soluble nucleotide precursor identified as UDP-NAM-pentapeptide by paper chromatography and quantitative amino acid analysis, indicating that the blocked step was the one catalyzed by MraY (FIG. 18). Moreover, when membrane samples were assayed for MraY activity using a UMP exchange assay, membranes from cells expressing E were found to be drastically reduced in MraY activity (FIG. 23). Also, a parallel exchange reaction assaying the activity of Rfe, which catalyzes the transfer of GlcNac to undecaprenolP, revealed that this reaction is unaffected during E expression (Table 2). Thus the inhibition of MraY by E is specific and does not extend to other undecaprenol-P dependent sugar transferases. Finally, quantitative amino acid analysis was used to show that the precursor accumulating in the E-inhibited cells was the pentapeptide precursor, the substrate for MraY (not shown). Taken together, these data constitute unequivocal proof that E inhibits MraY as its mode of lytic action (FIG. 18).

For cells expressing the A₂ lysis protein, cell wall synthesis, as assessed by labeling with ³H-DAP, was also blocked, and in fact degradation of the SDS-insoluble material was observed (FIG. 20). Again, no accumulation of label in the lipid-linked precursors was detected, but, in contrast to the E system, there was also no accumulation of the UDP-GlcNampentapeptide, demonstrating that the A₂ target was an early stepin precursor biosynthesis (FIG. 18).

### EXAMPLE 10

### Isolation of Bacteriophages that Target Cell Wall Synthesis

A multicopy plasmid carrying the target gene of a phage lysis protein that acts as a cell wall synthesis inhibitor can grossly delay the lytic action (FIG. 11). This leads to the concept that a straightforward method to find phages which target particular steps in cell wall biosynthesis is to construct a panel of bacterial strains with multicopy clones carrying one gene of the peptidoglycan biosynthesis pathway.

A panel of bacterial strains is assembled, each of which has one of the cell wall enzyme genes on a multicopy plasmid. Phages are isolated from the wild; for example, sewage or fecal matter. The liquid sample containing the phage is spotted on a lawn of bacteria growing on an agar plate. The plate is incubated overnight. The next day the plates are examined for plaques in the lawn. The initial lawn is the control lawn, with the multicopy plasmid vector but carrying no copy of a cell wall gene. Next, each plaque is then stabbed with a toothpick and then the virus-contaminated toothpick is stabbed into specific grid positions in new lawns, each made from one of the bacterial strains overexpressing one of the cell wall genes on a multicopy plasmid. Thus, with 20 different genes, there would be 20 different lawns. Then, after incubation, the plates are inspected for grid positions where there is no clearing zone or greatly reduced clearing zone, compared to the control lawn, indicating a plating defect. The grid positions on the control plate are used as sources for the candidate phage. Then, the candidate phage is suspected to target the cell wall gene that is on the multicopy plasmid; however, it was mutated. Using standard methods of molecular genetics, the lysis gene in the candidate phage is identified and sequenced similar to E or A₂ .

FIG. 11 illustrates this procedure using φX174. The lysis of the strain carrying the MraY plasmid is defective (absent or delayed). Thus, the many extra copies of the target gene product MraY protect against the phage lysis protein E from blocking the cell wall synthesis.

### EXAMPLE 11

### Isolation of Lysis Protein Genes with Mutations that Overcome Resistance

The procedure described in Example 9 can also be used to select for lysis polypeptides that overcome resistance mutations in the target gene. Proof of this is shown in FIG. 11. Here, it is shown that a multicopy plasmid carrying an allele of the target gene for a phage lysis protein (mraY4) can block the lysis event This will lead to loss of plaque-forming ability or reduced plaque size. This leads to the concept that a straightforward method to find phages which can overcome resistant target proteins is to select for mutant plaque-forming revertants by plating the phage on a lawn of a host carrying the resistance allele on its chromosome or on a multicopy plasmid. Alternatively, one can screen for enhanced plaque-size phages by plating out the phages on such lawns. If the frequency of revertant or enhanced plaque-size mutations is too low, then standard methods of mutagenesis can be applied to the phage stock before the selection or screening.

### EXAMPLE 12

### Development of Polypeptides that Mimic a Lysis Protein

A library of small polypeptide genes of random sequence are constructed by PCR amplification of a randomized synthetic DNA sequence carrying a fixed, efficient ribosome-binding site, start codon, and stop codon. This is inserted into a plasmid vector carrying an inducible promoter. Plasmids which cause inhibition of cell wall synthesis when induced are isolated by induction of this library, incubation under vigorous growth conditions for an extensive period, and then isolation of rare plasmid DNA is released as a result of a lytic polypeptide's action. Plasmid DNA is obtained in pure form by simply passing the culture filtrate through a DNA purification column and eluting the DNA that is bound.

This plasmid release protocol is repeated to enrich for positive clones. Each lytic sequence can be directly determined by PCR-based sequencing.

### REFERENCES CITED

All patents and publications mentioned in the specification are indicative of the level of those skilled in the art to which the invention pertains. All patents and publications are herein incorporated by reference to the same extent as if each individual publication was specifically and individually indicated to be incorporated by reference.
Bernhardt, T. G., Roof, W. D., and Young, R. (2000). Proc. Natl. Acad. Sci. U.S.A 97, 4297-4302.
Füirste, J. P., Pansegrau, W., Frank, R., Blöcker, H., Scholz, P., Bagdasarian, M. & Lanka, E. (1986) Gene 48, 119-131.
Hara, H., Yasuda, S., Horiuchi, K. and Park, J. T. (1997) J. Bacteriol. 179, 5802-5811.
Kleckner, N., Bender, J. & Gottesman, S. (1991) Methods Enzymol. 204, 139-180.
Maniatis, T., Fritsch, E. F. & Sambrook, J. (1982) Molecular Cloning: A Laboratory Manual (Cold Spring Harbor Lab. Press, Plainview, N.Y.).
Mengin-Lecreulx, D., Ayala, J., Bouhss, A., van Heijenoort, J., Parquet, C. and Hara, H. (1988) J. Bacteriol. 180, 4406-4412.
Miller, J. H. (1992) A Short Course in Bacterial Genetics: A Laboratory Manual and Handbook for Escherichia coli and Related Bacteria (Cold Spring Harbor Lab. Press, Plainview, N.Y.).
Roof, W. D., Fang, H. Q., Young, K. D., Sun, J. & Young, R. (1997) Mol. Microbiol. 25, 1031-1046.
Roof, W. D., Horne, S. M., Young, K. D. & Young, R. (1994) J. Biol. Chem. 269, 2902-2910.
Smith, D. L., Chang, C.-Y. & Young, R. (1998) Gene Exp. 7, 39-52.
Winter, R. B. and Gold, L. (1983). Cell 33, 877-885.

One skilled in the art readily appreciates that the present invention is well adapted to carry out the objectives and obtain the ends and advantages mentioned as well as those inherent therein. Systems, pharmaceutical compositions, treatments, methods, procedures and techniques described herein are presently representative of the preferred embodiments and are intended to be exemplary and are not intended as limitations of the scope. Changes therein and other uses will occur to those skilled in the art which are encompassed within the spirit of the invention or defined by the scope of the pending claims.

1. A method of screening for a candidate bacterial nucleic acid sequence that encodes a target polypeptide for a single-gene lysis polypeptide comprising:
contacting bacteria with a lysis polypeptide;
selecting for bacterial survivors of cell lysis caused by the lysis polypeptide that survive lysis by having a candidate bacterial nucleic acid sequence that encodes a target polypeptide making cells resistant to lysis by the lysis polypeptide; and
mapping the candidate bacterial nucleic acid sequence, wherein the mapped sequence corresponds to the nucleic acid sequence which encodes the target polypeptide.

2. The method of claim 1, wherein contacting the bacteria with the lysis polypeptide comprises transforming bacteria with a vector comprising a nucleic acid sequence that encodes a single-gene lysis polypeptide.

3. The method of claim 2, wherein contacting comprises inducing the expression of the lysis polypeptide.

4. The method of claim 1, wherein the lysis polypeptide is mutated.

5. The method of claim 1, further comprising isolating the mapped bacterial nucleic acid sequence.

6. The method of claim 5, further comprising determining the characteristics of the isolated bacterial nucleic acid sequence.

7. The method of claim 6, wherein determining the characteristics of the bacterial nucleic acid sequence comprises gel electrophoresis or nucleic acid sequence analysis.

8. The method of claim 1, further comprising inserting the mapped bacterial nucleic acid sequence in an expression vector to produce a polypeptide.

9. The method of claim 8, further comprising isolating the polypeptide.

10. The method of claim 9, further comprising determining the characteristics of the polypeptide.

11. The method of claim 10, wherein determining the characteristics comprises electrophoresis, spectrophotometric analysis, amino acid analysis, structural analysis or analysis of biochemical functions.

12. The method of claim 1, wherein the bacteria comprise a vector comprising a nucleic acid sequence encoding a polypeptide involved in cell wall synthesis.

13-50. (canceled)

51. The method of claim 1, wherein the bacterial nucleic acid sequence that encodes the target polypeptide is mraY or mura.

52. The method of claim 1, wherein the lysis polypeptide is E polypeptide or A₂ polypeptide.


SEQUENCE LISTING (2 sequences)

SEQ ID NO: 1 - protein, 91 aa, Bacteriophage phi-X174
MVRWTLWDTLAFLLLLSLLLPSLLIMFIPSTFKRPVSSWKALNLRKTLLMASSVRLKPLNCSRLPCVYAQETLTFLLTQKKTCVKNYVRKE

SEQ ID NO: 2 - protein, 419 aa, Escherichia coli
MDKFRVQGPTELQGEVTISGAKNAALPILFAALLAEEPVEIQNVPKLKDVDTSMKLLSQLGAKVERNGSVHIDARDVNVFCAPYDLVKTMRASIWALGPLVARFGQGQVSLPGGCTIGARPVDLHISGLEQLGATIKLEEGYVKASVDGRLKGAHIVMDKVSVGATVTIMCAATLAEGTTIIENAAREPEIVDTANFLITLGAKISGQGTDRIVIEGVERLGGGVYRVLPDRIETGTFLVAAAISRGKIICRNAQPDTLDAVLAKLRDAGADIEVGEDWISLDMHGKRPKAVNVRTAPHPAFPTDMQAQFTLLNLVAEGTGFITETVFENRFMHVPELSRMGAHAEIESNTVICHGVEKLSGAQVMATDLRASASLVLAGCIAEGTTVVDRIYHIDRGYERIEDKLRALGANIERVKGE